(19) United States
(12) Patent Application Publication
(10) Pub. No.: US 2022/0244252 A1
(43) Pub. Date: Aug. 4, 2022

(54) METHODS AND COMPOUNDS FOR TYPING RHEUMATOID FACTORS

(71) Applicant: Sanquin Innovatie B.V., Amsterdam (NL)

(72) Inventors: Taede RISPENS, Utrecht (NL); Willem Jan Julius FALKENBURG, Amsterdam (NL); Gerrit Jan WOLBINK, Amsterdam (NL); Dirkjan VAN SCHAARDENBURG, Hilversum (NL)

(21) Appl. No.: 17/617,199

(22) PCT Filed: Jun. 5, 2020
(86) PCT No.: PCT/NL2020/050370

§ 371 Date: Dec. 7, 2021

(30) Foreign Application Priority Data
Jun. 7, 2019 (EP) EP 19179099.7

(51) Int. Cl. G01N 33/564; G01N 33/68

(52) U.S. Cl. CPC G01N 33/564; G01N 33/6857; G01N 2800/52; G01N 2800/50; G01N 2800/102

(57) ABSTRACT

The invention relates to methods for typing rheumatoid factor comprising contacting a sample from a subject with at least one compound comprising a recombinant human IgG class fragment crystallizable (Fc) domain comprising first and second rheumatoid factor epitopes, to such compounds that can be used in the methods and to uses thereof.

### CROSS REFERENCE TO RELATED APPLICATIONS

This application is a U.S. National Phase of, and Applicant claims priority from, International Patent Application No. PCT/NL2020/050370, filed Jun. 5, 2020, which claims priority from EP 19179099, filed Jun. 7, 2019, each of which is incorporated herein by reference in its entirety.

### FIELD OF THE INVENTION

The invention relates to the typing of rheumatoid factors, in particular to identify clinically relevant distinguishable rheumatoid factors. The invention further relates to diagnosis of diseases or conditions characterized by the presence of rheumatoid factor.

### BACKGROUND OF THE INVENTION

Rheumatoid factors (RFs) were discovered in 1937 by Erik Waaler, who observed that sheep red blood cells sensitized with rabbit anti-sheep serum agglutinated after adding serum from a patient with rheumatoid arthritis (RA). They were later defined as autoantibodies that bind to other antibodies, specifically the constant domain (or fragment crystallizable (Fc)) of immunoglobulin G (IgG), and can be of any isotype. IgM RFs are the most extensively studied isotype and are present in about 70% of RA patients, with prevalence varying widely between studies, but are also found in other autoimmune conditions, in Waldenström's macroglobulinemia, during chronic infections such as hepatitis, and even at low frequency (increasing with age) in the healthy population. Testing for presence of RFs is standard practice in the diagnostic workup of patients suspected of RA and is included in the most recent ACR/EULAR RA classification criteria, as is testing for anti-citrullinated protein antibodies (ACPAs), the other major class of autoantibodies in RA.

Establishing RF status is not just important for diagnosing RA, but also for predicting development of RA in at-risk individuals and predicting disease course in RA patients. Surprisingly, despite its strong link to RA, a mechanism for how RF contributes to pathology has thus far not been established. A current hypothesis is that in the joint RFs enhance complement activation and production of pro-inflammatory cytokines by macrophages, through complex formation with ACPA-IgGs. In healthy individuals RFs may play a physiological role in binding and clearing immune complexes (ICs). These physiological RFs are thought to arise from so-called natural antibodies, ill-defined low affinity IgM antibodies that supposedly act as a first line of defense against various pathogens and reduce inflammation by clearing debris created by dead cells. Whether RFs contributing to pathology in the pre-clinical and clinical stages of RA arise from this pool of natural antibody-like RFs, or separately through an antigen driven immune response induced by ACPA-ICs, is currently unknown.

To measure RF's, commercially available RF assays used in the clinic measure agglutination of IgG coated particles by RF or detect IgM-RF with isotype specific antibodies. However, it is known that RFs are a heterogeneous pool of autoantibodies binding to multiple epitopes on the Fc region of human IgG. Studies have identified different RF reactivity patterns, for example by testing for reactivity against the four IgG subclasses, which differ slightly in their Fc region at the amino acid level. Specifically, IgG3-reactive RF responses were suggested to represent more pathogenic responses in RA. From analyzing single B cell clones isolated from RA patients, as well as healthy immunized donors (HIDs) and Waldenström's macroglobulinemia patients, it was found that RFs derived from RA patients more often showed ‘pan’ reactivity, i.e. reactivity towards all four subclasses. Furthermore, monoclonal RFs with similar overall binding characteristics, such as the ‘Ga.’ binding pattern, were found to display subtly different fine-specificities.

In other words, these studies indicate more heterogeneity in reactivity of RA-derived RFs, and suggest that if the RF response could be dissected into multiple individual reactivities, disease-specific RFs or patterns of RF reactivities might be identified. Despite its great potential, characterization of RF reactivity patterns directly in serum has thus far not been explored as a method to improve the clinical value of RF assays.

A major obstacle in doing so is the requirement of target molecules to which a single type of RF would bind at a time. Earlier studies attempted epitope mapping using linear peptide fragments (Peterson et al. 1995; Williams and Malone 1994), but such an approach cannot identify discontinuous epitopes, which require correctly folded proteins and which form the overwhelming majority of protein epitopes (Sivalingam and Shepherd 2012). For the analysis of RF repertoires directly in sera, individual IgG targets with correctly folded epitopes are required, to which only a specific portion of the RFs will bind.

From detailed binding studies of monoclonal RFs several individual molecular determinants have been identified as being important for RF binding, especially for the ‘Ga.’ reactivity pattern.

This was the first reactivity pattern described (Allen et al. 1966), characterized by binding of RF to IgG1, 2 and 4, with low or absent binding to IgG3. It was shown that the histidine (H) amino acid residue located at position 435 (H435) in IgG1, 2 and 4 is a crucial determinant in the epitopes bound by many (monoclonal) RFs with Ga reactivity (Bonagura et al. 1993). Other amino acids in the CH2 and/or CH3 domains of IgG were also found to be important for binding of monoclonal RFs showing a Ga binding pattern, and the contributing amino acids can differ between RFs (Bonagura et al. 1993). However, knowledge of a few determinants that are important for RF binding is in itself insufficient to arrive at a method to identify clinically relevant RF reactivity patterns.

Hence, there is a need in the art to provide means and methods to characterize RF reactivity patterns that can be directly applied to samples obtained from a patient.

### SUMMARY OF THE INVENTION

It is an object of the present invention to provide improved methods for typing RFs in a sample of a subject. Using such methods diseases-specific RF reactivity profiles can be established for disorders that are associated with the presence of RF using samples from patients suffering from the disorder. Such methods can further be used to establish RF reactivity profiles in samples from patients. It is a further object of the invention to provide compounds and combinations of compounds that can be used in such methods.

The invention therefore provides a method for typing rheumatoid factor (RF), comprising:
contacting a sample from a subject, said sample comprising or suspected of comprising RF, with at least one compound comprising a recombinant human IgG class fragment crystallizable (Fc) domain, wherein said Fc domain comprises:

1) a first RF epitope that is altered such that RF binding to said epitope is reduced with at least 20% as compared to binding to an unaltered first RF epitope and 4

2) a second RF epitope that is unaltered or that is altered such that RF binding to said epitope is reduced with at most 50% as compared to binding to an unaltered second RF epitope,

with the proviso that the percentage of reduction in binding to said first RF epitope is higher than the percentage of reduction in binding to said second RF epitope, and
determining binding of RF to said at least one compound.

In a further aspect, the invention provides a method for typing rheumatoid factor (RF), comprising:
contacting a sample from a subject, said sample comprising or suspected of comprising RF, with at least one compound comprising a recombinant human IgG class fragment crystallizable (Fc) domain, wherein said Fc domain comprises:

1) a first RF epitope wherein amino acid H435 and one or more amino acids selected from the group consisting of R255, L309, Q342, P343, E345, Y373, H433, T437, and Q438 of said IgG Fc domain are replaced by another amino acid, and

2) a second RF epitope comprising amino acid V422 and optionally one or more amino acids selected from the group consisting of R355, Q418, Q438, and P445 of said IgG Fc domain, or
wherein said compound comprises;

1) a first RF epitope wherein amino acid V422 and optionally one or more amino acids selected from the group consisting of R355, Q418, Q438, and P445 of said IgG Fc domain are replaced by another amino acid, and

2) a second RF epitope comprising amino acid H435 and one or more amino acids selected from the group consisting of R255, L309, Q342, P343, E345, Y373, H433, T437, and Q438 of said IgG Fc domain, or

wherein in said compound amino acids H435 and V422 and one or more amino acids selected from the group consisting of R255, Q342, P343, E345, Y373, H433 and T437 are replaced by another amino acid and the compound comprises one or more amino acids selected from the group consisting of R255, L309, Q342, P343, E345, Y373, H433, T437, R355, Q418 and P445, and
determining binding of RF to said at least one compound, wherein the amino acid numbering is according to EU numbering.

In a further aspect, the invention provides a combination of a first compound A and a second compound B, each compound comprising a human IgG class fragment crystallizable (Fc) domain, wherein said Fc domain comprises:

1) a first RF epitope that is altered such that RF binding to said epitope is reduced with at least 20% as compared to binding to an unaltered first RF epitope and

2) a second RF epitope that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, and wherein said second RF epitope in said first compound is different from said second RF epitope in said second compound.

In a further aspect, the invention provides a combination of a first compound A and a second compound B, each compound comprising a human IgG class fragment crystallizable (Fc) domain, wherein
compound A comprises:
   a first RF epitope wherein amino acid H435 and one or more amino acids selected from the group consisting of R255, L309, Q342, P343, E345, Y373, H433, T437, and Q438 of said IgG Fc domain are replaced by another amino acid, and
   a second RF epitope comprising amino acid V422 and optionally one or more amino acids selected from the group consisting of R355, Q418, Q438, and P445 of said IgG Fc domain, and
compound B comprises:
   a first RF epitope wherein amino acid V422 and optionally one or more amino acids selected from the group consisting of R355, Q418, Q438, and P445 of said IgG Fc domain are replaced by another amino acid, and
   a second RF epitope comprising amino acid V422 and optionally one or more amino acids selected from the group consisting of R355, Q418, Q438, and P445 of said IgG Fc domain.

In a further aspect, the invention provides a kit of parts comprising a combination according to the invention.

In a further aspect, the invention provides a compound comprising a recombinant human IgG class fragment crystallizable (Fc) domain, wherein said Fc domain comprises:

1) a first RF epitope that is altered such that RF binding to said epitope is reduced with at least 20% as compared to binding to an unaltered and wherein amino acids H435 and one or more amino acids selected from the group consisting of R255, L309, Q342, P343, E345, Y373, H433, T437 and Q438 of said IgG Fc domain are replaced by another amino acid, and

2) a second RF epitope that is unaltered or that is altered such that RF binding to said epitope is reduced with at most 50% as compared to binding to an unaltered second RF epitope and that comprises amino acid V422 and optionally one or more amino acids selected from the group consisting of R355, Q418 and P445 of said IgG Fc domain, with the proviso that the percentage of reduction in binding to said first RF epitope is higher than the percentage of reduction in binding to said second RF epitope, wherein said amino acid numbering is according to EU numbering.

In a further aspect, the invention provides a compound comprising a recombinant human IgG class fragment crystallizable (Fc) domain, wherein said Fc domain comprises:

1) a first RF epitope that is altered such that RF binding to said epitope is reduced with at least 20% as compared to binding to an unaltered first RF epitope and wherein amino acid V422 and optionally one or more amino acids selected from the group consisting of R355, Q418, Q438 and P445 of said IgG Fc domain are replaced by another amino acid, and

2) a second RF epitope that is unaltered or that is altered such that RF binding to said epitope is reduced with at most 50% as compared to binding to an unaltered second RF epitope and that comprises amino acid H435 and optionally one or more amino acids selected from the group consisting of R255, L309, Q342, P343, E345, Y373, H433, T437 and Q438 of said IgG Fc domain, with the proviso that the percentage of reduction in binding to said first RF epitope is higher than the percentage of reduction in binding to said second RF epitope, wherein said amino acid numbering is according to EU numbering.

In a further aspect, the invention provides a compound comprising a recombinant human IgG class fragment crystallizable (Fc) domain, wherein said Fc domain comprises:

1) a first RF epitope wherein amino acids H435 and one or more amino acids selected from the group consisting of R255, L309, Q342, P343, E345, Y373, H433, T437, and Q438 of said IgG Fc domain are replaced by another amino acid, and 2) a second RF epitope that comprises amino acid V422 and one or more amino acids selected from the group consisting of R355, Q418 and P445 of said IgG Fc domain, wherein said amino acid numbering is according to EU numbering.

In a further aspect, the invention provides a compound comprising a recombinant human IgG class fragment crystallizable (Fc) domain, wherein said Fc domain comprises:

1) a first RF epitope wherein amino acid V422 and optionally one or more amino acids selected from the group consisting of R355, Q418, Q438, and P445 of said IgG Fc domain are replaced by another amino acid, and

2) a second RF epitope that comprises amino acid H435 and one or more amino acids selected from the group consisting of R255, L309, Q342, P343, E345, Y373, H433 and T437 of said IgG Fc domain, wherein said amino acid numbering is according to EU numbering.

In a further aspect, the invention provides a compound comprising a recombinant human IgG class fragment crystallizable (Fc) domain, wherein in said compound amino acids H435 and V422 and one or more amino acids selected from the group consisting of R255, L309, Q342, P343, E345, Y373, H433 and T437 are replaced by another amino acid and the compound comprises one or more amino acids selected from the group consisting of R255, Q342, P343, E345, Y373, H433, T437, R355, Q418 and P445.

In a further aspect, the invention provides a compound comprising a recombinant human IgG class fragment crystallizable (Fc) domain, wherein said compound is a compound as described herein.

In a further aspect, the invention provides a use of a compound or combination of a first compound A and a second compound B according to the invention in diagnosis.

In a further aspect the invention provides a method for determining a RF reactivity profile characteristic for a disease or condition, preferably a disease or condition characterized by the presence of rheumatoid factor, comprising typing RFs in one or more samples, preferably a plurality of sample from patients suffering from the disease or condition with a method for typing RF according to the invention.

In a further aspect the invention provides a method for determining a treatment strategy for a subject, comprising typing RFs in a sample of the subject with a method for typing RF according to the invention, and determining a treatment strategy for said subject if said typing and/or the established RF reactivity profile indicates that said subject is suffering from or at risk of suffering from a particular disease or condition characterized by the presence of RF.

In a further aspect the invention provides a method of treatment of a subject in need thereof, comprising typing RFs in a sample of the subject using a method for typing RF according to the invention and providing said subject with treatment if said typing and/or the established RF reactivity profile indicates that said individual is suffering from or at risk of suffering from a particular disease or condition characterized by the presence of RF.

### DETAILED DESCRIPTION

The present inventors hypothesized that if the RF response could be dissected into multiple individual reactivities, disease-specific RFs or patterns of RF reactivities might be identified. For the analysis of RF repertoires in sera, individual, correctly folded epitopes are required, to which only a specific portion of the RFs will bind. A novel approach to break down the RF response and its epitopes in more detail, to identify clinically relevant RF binding patterns in patient cohorts is presented. For this goal, a human IgG molecule with hardly any residual RF binding was designed, which formed the basis for a set of targets to which RF binding was essentially confined to a specific region or epitope in the Fc domain. The present inventors found that mouse IgG2b has the least reactivity with human RF of all IgG types tested so far (see FIG. 2A). A human IgG molecule with hardly any residual RF binding was thus designed based on the difference in amino acid sequence with the mouse IgG2b molecule. By comparison of the human IgG1 and mouse IgG2b sequence the most relevant amino acids involved in RF binding could be identified. After having replaced the most crucial identified amino acids in human IgG1 with the amino acid at the corresponding position in the mouse IgG2b sequence or in the case of P445 with the amino acid at the corresponding position in IgG4, these amino acids were gradually reintroduced to identify relevant RF epitopes. In this way, the present inventors succeeded in providing IgG RF targets that can be used to evaluate directly in samples obtained from an individual his or her repertoire of polyclonal RF responses. The examples show that mutating three groups of predicted RF epitopes, comprising 16 amino acids in total, was sufficient to eliminate binding of a substantial part of the RF responses. Selective reversal of only a subset of these mutations generated a set of targets that made it possible to identify specific RF reactivity patterns in patients with either arthralgia, RA, or pSS. The approach presented here ultimately allows discrimination of RF responses to many different epitopes. Upon analyzing polyclonal RF responses in several patient cohorts, disease-specific RF specificities were identified. In particular, RF reaction patterns were identified that correlate with certain conditions or with a high or low risk of progression to disease. With the compositions, compounds and methods of the invention it has now become possible to identify such RF reaction patterns for instance in individuals suffering from or suspected of suffering from RA, Sjögren's syndrome or other diseases or conditions characterized by the presence of RFs and for instance in RF seropositive arthralgia patients to assess the risk of developing RA.

In a first aspect the invention therefore provides a method for typing rheumatoid factor (RF), comprising:
contacting a sample from a subject, said sample comprising or suspected of comprising RF, with at least one compound comprising a recombinant human IgG class fragment crystallizable (Fc) domain, wherein said Fc domain comprises: 1) a first RF epitope that is altered such that RF binding to said epitope is reduced with at least 20% as compared to an unaltered first RF epitope and

2) a second RF epitope that is unaltered or that is altered such that RF binding to said epitope is reduced with at most 50% as compared to an unaltered second RF epitope, with the proviso that the percentage of reduction in binding to said first RF epitope is higher than the percentage of reduction in binding to said second RF epitope, and
determining binding of RF to said at least one compound.

In a further aspect, the invention provides a combination of a first compound A and a second compound B, each compound comprising a human IgG class fragment crystallizable (Fc) domain, wherein said Fc domain comprises:

1) a first RF epitope that is altered such that RF binding to said epitope is reduced with at least 20% as compared to binding to an unaltered first RF epitope and

2) a second RF epitope that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, and wherein said second RF epitope in said first compound is different from said second RF epitope in said second compound. Further, preferably the first RF epitope in said first compound is different from the first RF epitope in said second compound.

The numbering of amino acids in IgG molecules as used herein is according to the EU numbering system (Edelman et al. 1969).

In amino acid sequences as used herein amino acids are denoted by single-letter symbols. These single-letter symbols and three-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine.

“Rheumatoid factor” (RF) is a term well known in the art and refers to autoantibodies of the IgM, IgG or IgA isotype, against the Fc portion of IgG antibodies. These autoantibodies are often present in RA patients, but also in in other autoimmune conditions.

The term “antibody” as used herein is well known in the art and refers to an immunoglobulin protein comprising at least one heavy chain variable region (VH), paired with a light chain variable region (VL), that is specific for a target epitope and at least one heavy chain constant region (CH) paired with a light chain constant region (CL).

The term “IgG” as used herein refers to an immunoglobulin IgG1, IgG2, IgG3 or IgG4, preferably to a human IgG1, IgG2, IgG3 or IgG4.

An “Fc-domain” a used herein refers to the part of an antibody heavy chain constant region beginning at the CH2 (residue 231 in IgG according to the EU numbering system) up to and including the C-terminus of the constant region.

Hence, an Fc domain comprises a CH2 domain and the CH3 domain.

A “CH2 domain” as used herein refers to a CH2 domain of an antibody. The CH2 domain includes amino acids 231 to 340 of a heavy chain immunoglobulin molecule (according to the EU numbering system).

A “CH3 domain” as used herein refers to a CH3 domain of an antibody. The CH3 domain includes amino acids 341 to 447 of a heavy chain immunoglobulin molecule (according to the EU numbering system).

As used herein “IgG class Fc domain” in the context of a domain or antibody indicates that the domain or antibody has the sequence of an Fc domain of an IgG1,

IgG2, IgG3 or IgG4 molecule, preferably of a human IgG1, IgG2, IgG3 or IgG4 domain. More preferably, an IgG class Fc domain as used herein is a human IgG1, IgG2 or IgG4 class Fc domain, more preferably a human IgG1 or IgG2 class Fc domain, most preferably a human IgG1 class Fc domain.

The terms “specifically binds” and “specific for” as used herein refer to the interaction between an antibody, or part thereof, and its epitope. The terms means that said antibody, or part thereof, preferentially binds to said epitope over other amino acid sequences or portions of the antigen or over other antigens. Although the antibody or part may non-specifically bind to other portions, amino acid sequences or antigens, the binding affinity of said antibody or part for its epitope is significantly higher than the non-specific binding affinity of said antibody or part for other portions, amino acid sequences or antigens. In this context the term “reactivity” is also used. Reactivity of RFs refers to the ability of RFs to specifically bind to an epitope.

“Binding affinity” refers to the strength of the total sum of the noncovalent interactions between a single binding site of an antibody or functional part or functional equivalent and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity can generally be represented by the equilibrium dissociation constant (KD), which is calculated as the ka to kd ratio, see, e.g., Chen, Y., et al., (1999) J. Mol Biol 293:865-881. Affinity can be measured by common methods known in the art, such as for instance a Surface Plasmon Resonance (SPR) assay such as BiaCore (GE Healthcare) or IBIS-iSPR instrument at IBIS Technologies BV (Hengelo, the Netherlands) or solution phase assays, such as Kinexa.

The term “sample” as used herein refers to a sample obtained from an individual. In particular, as used herein a sample refers to a sample in which RF are present if an individual has RF. In a preferred embodiment said sample is a body fluid. In a further preferred embodiment said sample is a blood product sample or a synovial fluid sample. Most preferably said sample is a blood product sample. The term “blood product” as used herein refers to a product that comprises one or more blood components, such as plasma or serum, and/or whole blood. Preferably, the blood product is plasma, serum or whole blood, more preferably serum.

As used herein, the term “subject” encompasses humans and animals, preferably mammals. Preferably, a subject is a mammal, more preferably a human. In a particular embodiment, a subject comprises or is suspected of comprising RF. In a further preferred embodiment, the subject is suffering from or suspected of suffering from a disease or condition characterized by the presence of RF. Non-limiting preferred examples of such disease or condition are rheumatoid arthritis (RA), arthralgia, systemic lupus erythematosus (SLE), Sjögren syndrome, interstitial pulmonary fibrosis, hepatitis B, chronic liver disease, and chronic hepatitis, essential mixed cryoglobulinemia, primary biliary cirrhosis, infectious mononucleosis and any chronic viral infection, bacterial endocarditis, leprosy, sarcoidosis, tuberculosis, syphilis, visceral leishmaniasis, malaria, leukemia, dermatomyositis and systemic sclerosis. In a preferred embodiment said disease or condition is selected from the groups consisting of RA, arthralgia and Sjögren syndrome.

RFs form a heterogenic population of autoantibodies and different RFs can recognize different parts of the Fc domain of IgG. The present invention make use of at least two different parts of the Fc domain of IgG that can be bound by different RF's to type, characterize or distinguish different RFs. The different parts of the Fc domain which can be recognized by different RFs are herein referred to as the “Elbow region (ER) epitope”, and the “Tail epitope”. The present inventors have further identified a third part of the Fc domain of IgG that can be bound by different RF's, which is herein referred to as the “CH2 epitope”.

One epitope that can be recognized by RFs comprises amino acids at positions 433, 435 and 437 and optionally at positions 255, 309, 342, 343, 345 and 373 of the IgG class Fc domain (according to EU numbering), for instance amino acids R255, L309, Q342, P343, E345, Y373, H433, H435 and/or T437 in a human IgG1, IgG2 and IgG4. This RF epitope is herein also referred to as the “ER epitope”.

Another epitope that can be recognized by RFs comprises amino acids at positions amino acids at positions 355, 418 and 422 and optionally at position 445 of the IgG class Fc domain, for instance amino acids R355, Q418, V422 and/or P445 or L445 in a human IgG1, IgG2 and IgG4. This RF epitope is herein also referred to as the “Tail epitope”.

Yet another epitope that can be recognized by RFs comprises amino acids at positions amino acids at positions 278, 292 and 293 of the IgG class Fc domain, for instance amino acids Y278, R292 and E293 in a human IgG1, IgG2 and IgG4. This RF epitope is herein also referred to as the “CH2 epitope”. The term epitope is well known to a skilled person in the art of antibodies and refers to a portion of an antigen to which an antibody specifically binds. An epitope is not determined solely by amino acids that interact with the antibody, but also by other amino acids that do not interact with the antibody but that affect the ability of the amino acids that do interact with the antibody to adopt the 3-D conformation of the epitope. Such other amino acids may be contained within the 3D structure of the epitope or may be more distant, such that they are not contained within the 3-D yet still affect the 3-D conformation of the epitope. In the context of the present invention, the term “RF epitope” refers to a portion of an Fc domain to which a particular RF specifically binds and it includes both amino acids that interact with said RF and amino acids present in said portion that do not interact with said RF but that affect the ability of the amino acids that do interact with said RF to adopt the 3-D conformation of the epitope. An RF epitope of an Fc domain as used herein consists thus of surface exposed moieties of amino acids and optionally of other moieties, e.g. saccharide chains, and has specific three-dimensional structural properties. An RF epitope can be composed of contiguous and discontiguous amino acids. Hence, an RF epitope is typically formed of amino acids from different portions of the linear sequence of an Fc domain which are in close proximity in the tertiary or three-dimensional structure of the Fc domain. Such epitope is also referred to as a conformational epitope.

Hence, a skilled person understands that an RF epitope comprising one or more amino acids at positions 255, 309, 342, 343, 345, 373, 433, 435 and 437 of an IgG class Fc domain (i.e. the “ER epitope”) refers to a portion of the Fc domain formed by at least these one or more amino acids that are spatially close to each other in the three-dimensional structure of the Fc domain Hence, said epitope is characterized by said one or more amino acids, but may comprises further amino acids. In one embodiment said epitope comprises or consists of amino acids at positions 255, 309, 342, 343, 345, 373, 433, 435 and 437 of IgG class Fc domain.

Similarly, a skilled person understands that an RF epitope comprising one or more amino acids at positions 355, 418, 422 and 445 of an IgG class Fc domain (i.e. the “Tail epitope”) refers to a portion of the Fc domain formed by at least these one or more amino acids that are spatially close to each other in the three-dimensional structure of the Fc domain. Hence, said epitope is characterized by said one or more amino acids, but may comprises further amino acids. In one embodiment, said epitope comprises or consists of amino acids at positions 355, 418, 422 and 445 of IgG class Fc domain.

Similarly, a skilled person understands that an RF epitope comprising amino acids at positions Y278, R292 and E293 of IgG class Fc domain (i.e. the “CH2 epitope”) refers to a portion of the Fc domain formed by at least these amino acids that are spatially close to each other in the three-dimensional structure of the Fc domain. Hence, said epitope is characterized by said amino acids, but may comprises further amino acids. Preferably said epitope comprises or consists of amino acids at positions Y278, R292 and E293 of IgG class Fc domain.

The percentage of identity of an amino acid sequence, or the term “% sequence identity”, is defined herein as the percentage of residues of the full length of an amino acid sequence that is identical with the residues in a reference amino acid sequence or after aligning the two sequences and introducing gaps, if necessary, to achieve the maximum percent identity. Methods and computer programs for the alignment are well known in the art, for example “Align 2”. Programs for determining nucleotide sequence identity are also well known in the art, for example, the BESTFIT, FASTA and GAP programs. These programs are readily utilized with the default parameters recommended by the manufacturer.

A method for typing RF according to the invention comprises contacting a sample, preferably a blood product, of a subject with at least one compound comprising a recombinant human IgG class Fc domain according to the invention and determining binding of RF to said at least one compound. Said compound comprises at least an Fc domain of a human IgG because RF's binds to the Fc domain of IgG. The compound thus comprises at least an IgG CH2 and CH3 domain or a pFc′ region. Preferably the compound comprises an Fc region, i.e. the CH2 and CH3 domains and at least part of the hinge region. In a particular embodiment, the compound consists of the Fc region. However, the compound may further comprise a CH1 domain, a light chain constant domain, a light chain variable domain and/or a heavy chain variable domain or parts thereof. In one preferred embodiment, the compound is an IgG class molecule, preferably an IgG1, IgG2 or IgG4, more preferably an IgG1 or IgG2, most preferably an IgG1. As used herein, a “human IgG class Fc domain” means that the amino acids sequence of the Fc domain is at least 80% identical to the sequence of a naturally occurring human IgG molecule, preferably at least 85%, more preferably at least 90%, more preferably at least 95%, more preferably at least 98%, more preferably at least 99% identical to a naturally occurring human IgG molecule, as long as the requirements for the first and second, and optionally third, RF epitope as defined herein are met. In a particular embodiment, said human IgG class Fc domain is essentially identical or identical to a human IgG class molecule, with the exception of the alterations in the first and optionally second and/or third RF epitopes as defined herein. Said human IgG molecule is preferably a human IgG1, IgG2 or IgG4, more preferably a human IgG1 or IgG2, most preferably a human IgG1. Modifications may for instance be made to increase stability of the compound or to increase binding of RF to one of the epitopes. In one embodiment a compound according to the invention consists of the human IgG class Fc domain, preferably an Fc region or IgG molecule. However, the compound according to or used in accordance with the invention may comprise further moieties, such as a detectable label to enable detection of binding of RF to the compound. In a preferred embodiment, the compound according to or used in accordance with the invention comprises a detectable label. In a particular embodiment, the compound according to or used in accordance with the invention consists of a human IgG class Fc domain (such as an Fc region or IgG molecule) and a detectable label. Non-limiting examples of a detectable label are a fluorescent label, a luminescent label, a (radio)isotope label, a paramagnetic label, a (bio)nanoparticle label, a combination of two or more of said labels and a hybrid thereof. Such labels are well known in the art and a skilled person is well capable of identifying a suitable label for detection of binding of RF to the compound.

In the present invention one of the RF epitopes (the “first RF epitope”) is modified such that binding of RF to this epitope is reduced. Such compound can be used to determine whether a sample comprises RFs that bind to this epitope, e.g. by comparing binding of RFs in the sample to this compound with binding of RFs in the sample to a compound wherein this RF epitope is unaltered. In one embodiment only one RF epitope is modified to reduce binding of RF to said epitope and that other RF epitopes are unaltered. However, it is also possible to modify a second and/or third RF epitope, as long the percentage of reduction in binding to said first RF epitope is higher than the percentage of reduction in binding to said second RF epitope. The Fc domain of the compound thus comprises:

1) a first RF epitope that is altered such that RF binding to said epitope is reduced with at least 20% as compared to an unaltered first RF epitope and

2) a second RF epitope that is unaltered or that is altered such that RF binding to said epitope is reduced with at most 50% as compared to an unaltered second RF epitope, with the proviso that the percentage of reduction in binding to said first RF epitope is higher than the percentage of reduction in binding to said second RF epitope.

In other words, the Fc domain in a compound according to, or used in accordance with, the invention comprises:
a first RF epitope that is altered such that RF binding to said epitope is reduced with x % as compared to an unaltered first RF epitope, wherein x is at least 20, and
a second RF epitope that is unaltered or that is altered such that RF binding to said epitope is reduced with y % as compared to an unaltered second RF epitope, wherein y is at most 50, with the proviso that x>y.

It is within the capability of a skilled person to determine a suitable reduction of binding to the first RF epitope and optionally the second and/or third epitope to allow typing of RF on the basis of binding to at least one RF epitope. It is preferred that the percentage of reduction in binding to said first RF epitope is at least twice the percentage of reduction in binding to said second RF epitope, or, in other words, that x>2·y.

The first and second RF epitopes in a compound according to, or used in accordance with, the invention are different. The Fc domain may comprise a third RF epitope. In that case, this third RF epitope is different from the first and second RF epitopes and is preferably altered such that RF binding to said epitope is reduced with at least 20% as compared to an unaltered third RF epitope, with the proviso that the percentage of reduction in binding to said third RF epitope is higher than the percentage of reduction in binding to said second RF epitope. In this aspect, he Fc domain of the compound thus comprises:

1) a first RF epitope that is altered such that RF binding to said epitope is reduced with at least 20% as compared to an unaltered first RF epitope and a second RF epitope that is unaltered or that is altered such that RF binding to said epitope is reduced with at most 50% as compared to an unaltered second RF epitope

2) a third RF epitope that is altered such that RF binding to said epitope is reduced with at least 20% as compared to binding to an unaltered third RF epitope, with the provision that the percentage of reduction in binding to said first epitope and the percentage of reduction in binding to said third RF epitope are both higher than the percentage of reduction in binding to said second RF epitope.

In other words, the Fc domain in a compound according to, or used in accordance with, the invention in this aspect comprises:

1) a first RF epitope that is altered such that RF binding to said epitope is reduced with x % as compared to an unaltered first RF epitope, wherein x is at least 20,

2) a second RF epitope that is unaltered or that is altered such that RF binding to said epitope is reduced with y % as compared to an unaltered second RF epitope, wherein y is at most 50, and

3) a third RF epitope that is altered such that RF binding to said epitope is reduced with z % as compared to an unaltered third RF epitope, wherein z is at least 20, and with the proviso that x>y and z>y.

It is preferred that the percentage of reduction in binding to said first and third RF epitopes each are at least twice the percentage of reduction in binding to said second RF epitope, or, in other words, that x>2·y and z>2·y.

Alternatively the third RF epitope, preferably comprising amino acids at positions 278, 292 and 293, is unaltered or altered such that RF binding to said epitope is reduced with at most 50% as compared to binding to an unaltered second RF epitope, with the proviso that the percentage of reduction in binding to said first RF epitope is higher than the percentage of reduction in binding to said third RF epitope.

As used herein “altered” means that at least one of the amino acids comprised by the relevant RF epitope is altered as compared to the corresponding amino acid in a naturally occurring human IgG Fc domain. Alteration as used herein preferably is a replacement by another amino acid or deletion of the amino acid, more preferably replacement by another amino acid. A skilled person is well able to determine the amino acid alterations that result in a reduction of binding of RF to the RF epitope. For instance, binding of a specific RF to an Fc domain with an altered amino acid can be compared with binding of the same RF to an otherwise identical Fc domain with one or more altered, preferably replaced, amino acids within the RF epitope using a method as described herein below.

“Unaltered” as used herein means that the amino acids present in the RF epitope are identical to amino acids at the corresponding position in a naturally occurring human IgG class Fc domain or IgG molecule, preferably an IgG1, IgG2 or IgG4, more preferably an IgG1 or IgG2, most preferably an IgG1.

“Reduced binding” as used herein means that reactivity of a population of RFs containing RFs that are able to specifically bind to a particular epitope with that epitope that is altered in accordance with the invention is less than reactivity of the same population of RFs with the same epitope that is unaltered, for instance because less of the RFs in the population bind to the epitope. This can for instance be determined by comparing binding of RFs in a part of a sample to a compound comprising a first and second RF epitope as defined herein with binding of RFs in another part of the same sample to a reference compound. For instance, a sample comprising pooled RFs, including RFs that are able to specifically bind to the particular epitope, from multiple individuals can be used to determine whether binding of RF to the epitope is reduced in a compound according to the invention or used in accordance with the invention. For instance, samples, such as blood samples, of e.g. 5, 10, 15, 20 or 50 individuals can be pooled to assess whether binding of RF to a specific altered epitope is reduced and to which extent. It is within the capabilities of a skilled person to select a suitable method to detect or measure binding of RFs to a compound as defined herein and a reference compound and to select a suitable reference compound.

A suitable reference compound is a compound that is identical to the compound as defined herein with the exception that it contains an unaltered first RF epitope. Another suitable reference compound is a compound that is identical to the compound as defined herein with the exception that it contains both an unaltered first epitope and an unaltered second RF epitope and optionally an unaltered third RF epitope.

A suitable method to detect or measure binding of RFs to a compound according to the invention is described in detail in the examples herein. In brief, RF reactivity against the compound can be analyzed in enzyme-linked immunosorbent assays (ELISAs), whereby the compound can be coated on 96-wells flat-bottom plates and, after appropriate washing steps, addition of the sample comprising RF, and optionally controls or reference samples, IgM-RF can be detected by incubating the wells with, e.g. horseradish peroxidase (HRP)-conjugated mouse monoclonal, anti-human IgM (μ-chain-specific) antibodies and visualized with 3,3′,5,5′-tetramethylbenzidine (100 μg/mL) followed by optical density (OD) reading at 450 nm and 540 nm for background correction. Levels of IgM-RF can be calculated using a tailor-made calibrator curve or, as in the examples using a calibrator curve of a reference serum used in a conventional IgM-RF ELISA (Klein et al. 1987).

Another suitable method to detect or measure binding of RFs to a compound according to the invention is an agglutination test. In such test, for instance the compound or compounds according to the invention are immobilized on beads or cells and contacted with a sample comprising or suspected of comprising RF, after which the sample can be assessed by visual inspection or light scattering and/or light absorption. A semi-quantitative result can be obtained by testing serial dilutions of a sample. Similarly as for ELISA, levels of IgM-RF can be calculated using a tailor-made calibrator curve or using a calibrator curve of a reference serum.

In a compound according to the invention or used in a method according to the invention the first RF epitope is altered such that RF binding to said epitope is reduced with at least 20%, preferably at least 30%, more preferably with at least 40%, more preferably with at least 50%, more preferably with at least 60%, more preferably with at least 70%, more preferably with at least 80%, more preferably with at least 90% as compared to an unaltered first RF epitope. In one embodiment, said first RF epitope is altered such that RF binding to said epitope is essentially absent as compared to binding to an unaltered first RF epitope.

In a compound according to or used in accordance with the invention the second RF epitope is altered such that RF binding to said epitope is reduced with at most 50%, preferably with at most 40%, more preferably with at most 25%, such as with at most 20%, such as with at most 15%, such as with at most 10%, such as with at most 5% as compared to an unaltered second RF epitope. In a particular embodiment, said second RF epitope is unaltered.

In a compound according to or used in accordance with the invention a third RF epitope is optionally altered such that RF binding to said epitope is reduced with at least 20%, preferably at least 30%, more preferably with at least 40%, more preferably with at least 50%, more preferably with at least 60%, more preferably with at least 70%, more preferably with at least 80%, more preferably with at least 90% as compared to an unaltered third RF epitope. In one embodiment, said third RF epitope is altered such that RF binding to said epitope is essentially absent as compared to binding to an unaltered third RF epitope.

The compounds comprising a human IgG class Fc domain according to the invention or used in a method of the invention may comprise multiple RF epitopes of which one is preferably unaltered or slightly altered to reduce RF binding with at most 50%, and the other epitope or epitopes are altered to reduce binding by RF with at least 20%. As a result RFs can bind to the unaltered or slightly altered epitope but not or in a substantially less degree to the other epitope(s). In one aspect one of such compound is used in a method of the invention. This may provide sufficient information for typing RF, for instance if information regarding binding to the first epitope is sufficient. If multiple of such compounds are used in which different epitopes are altered to reduce binding of RF with at least 20%, it is possible to distinguish RFs on the basis of the epitope they bind. This way it is possible to determine to which epitope(s) RF's that are present in a sample bind. Hence, in a preferred embodiment at least two of such compounds are used in a method of the invention, and preferably the first epitope that is altered such that RF binding to said epitope is reduced with at least 20% is different in both compounds. Provided is therefore a method according to the invention comprising contacting the sample from the subject with at least two of said compounds comprising a recombinant human IgG class Fc domain, wherein said second RF epitope in each of said two compounds is different, the method further comprising determining binding of RF to said at least two compounds. For instance, if two of such compounds are used, the first and second RF epitopes can for instance be swapped. I.e. the first RF epitope in a first compound A is the second RF epitope in a second compound B and the second RF epitope in the first compound A is the first RF epitope in the second compound B. It should be understood that as many different compounds can be used in a method of the invention, whereby said method comprises contacting the sample with each of said compounds and determining for each of said compounds binding of RF to the compound.

In another aspect, the invention provides a combination of a first compound A and a second compound B, each compound comprising a human IgG class fragment crystallizable (Fc) domain, wherein said Fc domain comprises:

1) a first RF epitope that is altered such that RF binding to said epitope is reduced with at least 20% as compared to binding to an unaltered first RF epitope and

2) a second RF epitope that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, and wherein said second RF epitope in said first compound is different from said second RF epitope in said second compound. Further, preferably the first RF epitope in said first compound is different from the first RF epitope in said second compound. Compounds A and B of a combination according to the invention preferably comprise a recombinant human IgG1, IgG2 or IgG4 class Fc domain, preferably a recombinant human IgG1.

Also provided is a kit of parts comprising the combination according to the invention. Such kit of parts is suitable for performing a method for typing RF according to the invention. In one embodiment, a kit of parts comprises two or more containers, whereby a first container comprises compound A and a second container comprises compound B. Associated with such containers can be various written materials such as instructions for use, or a notice in the form prescribed by a governmental agency regulating the manufacture, use or sale of diagnostic products. Preferably, a kit of parts according to the invention comprises instructions for use.

In a further aspect the invention provides a compound comprising a recombinant human IgG class fragment crystallizable (Fc) domain, wherein said Fc domain comprises:

1) a first RF epitope that is altered such that RF binding to said epitope is reduced with at least 20% as compared to binding to an unaltered and wherein amino acids H435 and one or more amino acids selected from the group consisting of R255, L309, Q342, P343, E345, Y373, H433, T437 and Q438 of said IgG Fc domain are replaced by another amino acid, and

2) a second RF epitope that is unaltered or that is altered such that RF binding to said epitope is reduced with at most 50% as compared to binding to an unaltered second RF epitope and that comprises amino acid V422 and optionally one or more amino acids selected from the group consisting of R355, Q418 and P445 of said IgG Fc domain, with the proviso that the percentage of reduction in binding to said first RF epitope is higher than the percentage of reduction in binding to said second RF epitope, wherein said amino acid numbering is according to EU numbering.

In a further aspect, the invention provides a compound comprising a recombinant human IgG class fragment crystallizable (Fc) domain, wherein said Fc domain comprises:

1) a first RF epitope that is altered such that RF binding to said epitope is reduced with at least 20% as compared to binding to an unaltered first RF epitope and wherein amino acid V422 and optionally one or more amino acids selected from the group consisting of R355, Q418, Q438 and P445 of said IgG Fc domain are replaced by another amino acid, and

2) a second RF epitope that is unaltered or that is altered such that RF binding to said epitope is reduced with at most 50% as compared to binding to an unaltered second RF epitope and that comprises amino acid H435 and optionally one or more amino acids selected from the group consisting of R255, L309, Q342, P343, E345, Y373, H433, T437 and Q438 of said IgG Fc domain, wherein said amino acid numbering is according to EU numbering.

A compound according to the invention preferably comprises a recombinant human IgG1, IgG2 or IgG4 class Fc domain, preferably a recombinant human IgG1.

In a preferred method or combination of compounds according to the invention the first RF epitope comprises amino acids at position 435, 433 and 437, or amino acids at positions 435, 255 and 309, or amino acids at positions 435, 342, 343, 345 and 373, or amino acids at positions 435, 433, 437, 342, 343, 345 and 373 of said Fc domain, and said second RF epitope comprises amino acids at position 422 and optionally at positions 355, 418 and/or 445 of said Fc domain. In an alternative preferred embodiment, the first and second epitope are switched. I.e. in an alternative preferred embodiment the first RF epitope comprises amino acids a positions 422 and optionally at positions 355, 418 and/or 445 of said Fc domain and said second RF epitope comprises amino acids at positions 435, 433 and 437, or amino acids at positions 435, 255 and 309, or amino acids at positions 435, 342, 343, 345, or amino acids at positions 435, 433, 437, 342, 343, 345 and 373 of said Fc domain.

In one particularly preferred method or combination of compounds according to the invention the first RF epitope in a compound comprises amino acids at positions 433, 435 and 437 and optionally one or more amino acids at positions 255, 309, 342, 343, 345, 373 and/or 438 of the Fc domain and the second RF epitope in said compound comprises amino acids at position 355, 418 and 422 and optionally at position 438 and/or 445 of the Fc domain. In an alternative preferred embodiment, the first and second epitope are switched. I.e. in an alternative preferred embodiment the first RF epitope comprises amino acids a positions 355, 418 and 422 and optionally at position 438 and/or 445 of the human IgG Fc domain and the second RF epitope comprises amino acids at positions 433, 435 and 437 and optionally amino acids at positions 255, 309, 342, 343, 345, 373 and/or 438 of the human IgG Fc domain.

In another particularly preferred method or combination of compounds according to the invention the first RF epitope in a compound comprises amino acids at positions 255, 309, 342, 343, 345, 373, 433, 435 and 437 of the Fc domain and the second RF epitope in said compound comprises amino acids at positions 355, 418, 422 and 445 of the Fc domain. In an alternative preferred embodiment, the first and second epitope are switched.

I.e. in an alternative preferred embodiment the first RF epitope comprises amino acids a positions 355, 418, 422 and 445 of the human IgG Fc domain and the second RF epitope comprises amino acids at positions 255, 309, 342, 343, 345, 373, 433, 435 and 437 of the human IgG Fc domain.

If at least two compounds are used in a method of the invention and in a combination according to the invention, it is preferred that:
a first compound A comprises:

a first RF epitope comprising amino acids at positions 435, 433 and 437, or amino acids at positions 435, 255 and 309, or amino acids at positions 435, 342, 343, 345 and 373, or amino acids at positions 435, 433, 437, 342, 343, 345 and 373 of said Fc domain that is altered such that RF binding to said epitope is reduced with at least 20% as compared to binding to an unaltered first RF epitope, and

a second RF epitope comprising amino acids at position 422 and optionally at positions 355, 418 and/or 445, preferably at positions 355, 418, 422 and 445, of said Fc domain that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, and
a second compound B comprises:

a first RF epitope comprising amino acids at positions 422 and optionally at positions 355, 418 and/or 445, preferably at positions 355, 418, 422 and 445, of said IgG Fc domain that is altered such that RF binding to said epitope is reduced with at least 20% as compared to binding to an unaltered first RF epitope, and

a second RF epitope comprising amino acids at positions 435, 433 and 437, or amino acids at positions 435, 255 and 309, or amino acids at positions 435, 342, 343, 345 and 373, or amino acids at positions 435, 433, 437, 342, 343, 345 and 373 of said Fc domain that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope.

In one preferred embodiment:
a first compound A comprises:

a first RF epitope comprising amino acids at positions 433, 435 and 437 and optionally amino acids at positions 255, 309, 342, 343, 345, 373 and/or 438 of said Fc domain that is altered such that RF binding to said epitope is reduced with at least 20% as compared to binding to an unaltered first RF epitope, and

a second RF epitope comprising amino acids at position 355, 418, 422 and 445 of said Fc domain that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, and
a second compound B comprises:

a first RF epitope comprising amino acids at positions 355, 418, 422 and 445, and optionally 438 of said Fc domain that is altered such that RF binding to said epitope is reduced with at least 20% as compared to binding to an unaltered first RF epitope, and

a second RF epitope comprising amino acids at positions 433, 435 and 437 and optionally amino acids at positions 255, 309, 342, 343, 345 and/or 373 of said Fc domain that is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope.

The first or second RF epitope comprising amino acids at position 355, 418 and 422 and optionally at position 445 of the human IgG Fc domain in one preferred embodiment comprises amino acids at position 355, 418, 422 and 445 of the IgG Fc domain. The first or second RF epitope comprising amino acids at positions 433, 435 and 437 and optionally amino acids at positions 255, 309, 342, 343, 345 and/or 373 of the human IgG Fc domain in one preferred embodiment comprises amino acids at positions 255, 309, 342, 343, 345, 373, 433, 435 and 437 of the human IgG Fc domain.

A third RF epitope that is altered such that RF binding to said epitope is reduced with at least 20% as compared to binding to an unaltered third RF epitope, with the proviso that the percentage of reduction in binding to said third RF epitope is higher than the percentage of reduction in binding to said second RF epitope, preferably comprises amino acids at positions Y278, R292 and E293.

Which of the amino acids that are present in each of the epitopes can be altered, preferably replaced by another amino acid, in order to reduce binding by at least 20% or to reduce binding by at most 50% can be readily determined by a person skilled in the art by synthesizing the compound with altered amino acids and testing binding of RFs to this compound as compared to a similar compound with unaltered first and second RF epitopes, for instance using a method as described herein.

In a preferred compound or first compound A according to, or used in accordance with, the invention, in the first RF epitope amino acid H435 and optionally one or more amino acids selected from the group consisting of R255, L309, Q342, P343, E345, Y373, H433, T437 and Q438 of said IgG Fc domain are replaced by another amino acid and the second RF epitope comprises amino acid V422 and optionally one or more amino acids selected from the group consisting of R355, Q418 and P445 of the human IgG Fc domain. As used herein the phrase As used herein the phrase “n the first RF epitope amino acid X123 is replaced by another amino acid” means that in said compound any amino acid can be present at amino acid position 123, except for X. For instance, “in the first RF epitope amino acid H435 is replaced by another amino acid” means that in said compound any amino acid can be present at amino acid position 435, except for histidine (H). Similarly, “optionally one or more amino acids selected from the group consisting of R255, L309, Q342, P343, E345, Y373, H433, T437 and Q438 of said IgG Fc domain are replaced by another amino acid” means that in said compound in 1, 2, 3, 4, 5, 6, 7, 8 or all of the indicated amino acid positions an amino acid is present that is different than the amino acid indicated for that position. As used herein the phrase “the second RF epitope comprises amino acid X123” means that in said compound X is present at amino acid position 123. For instance, “the second RF epitope comprises amino acid V422” means that in said compound a valine (V) is present at amino acid position 422. It is further preferred that said compound comprises a first RF epitope wherein amino acid H435 and one or more amino acids selected from the group consisting of R255, L309, Q342, P343, E345, Y373, H433, T437, and Q438 of said IgG Fc domain are replaced by another amino acid and a second RF epitope comprising amino acid V422, and optionally one or more amino acids selected from the group consisting of R355, Q418, and P445 of said IgG Fc domain. In a preferred compound according to the invention or used in a method of the invention (“compound A1”), amino acids H433, H435 and T437 are replaced by another amino acid and optionally one or more amino acids selected from the group consisting of R255, L309, Q342, P343, E345 and Y373 are replaced by another amino acid.

In yet another preferred compound according to the invention or used in a method of the invention (“compound A2”), amino acids H433, H435 and T437 are replaced by another amino acid and the compound comprises amino acids R255, L309, Q342, P343, E345 and Y373.

In yet another preferred compound according to the invention or used in a method of the invention (“compound A3”), amino acids R255, L309 and H435, are replaced by another amino acid and the compound comprises amino acids Q342, P343, E345, Y373, H433, and T437.

In yet another preferred compound according to the invention or used in a method of the invention (“compound A4”), amino acids Q342, P343, E345, Y373 and H435 are replaced by another amino acid and the compound comprises amino acids R255, L309, H433 and T437.

In yet another preferred compound according to the invention or used in a method of the invention (“compound A5”), amino acids R255, L309, H433 H435 and T437 are replaced by another amino acid and the compound comprises amino acids Q342, P343, E345 and Y373.

In yet another preferred compound according to the invention or used in a method of the invention (“compound A6”), amino acids R255, L309, Q342, P343, E345, Y373, H433, H435 and T437 are replaced by another amino acid.

In all these compounds A and A1 to A6 it is further preferred that the second epitope that is unaltered or that is altered such that RF binding to said epitope is reduced with at most 50% as compared to binding to an unaltered second RF epitope comprises amino acids R355, Q418, V422 and P445.

In another preferred compound, or second compound B according to, or used in accordance with the invention, in the first RF epitope amino acid V422 and optionally one or more amino acids selected from the group consisting of R355, Q418, Q438 and P445 of said IgG Fc domain are replaced by another amino acid and the second RF epitope comprises amino acid H435 and optionally one or amino acids selected from the group consisting of R255, L309, Q342, P343, E345, Y373,

H433 and T437 of said IgG Fc domain. It is further preferred that said compound comprises a first RF epitope wherein amino acid V422 and one or more amino acids selected from the group consisting of R355, Q418, Q438, and P445 of said IgG Fc domain are replaced by another amino acid and a second RF epitope comprising amino acid H435 and optionally one or more amino acids selected from the group consisting of R255, L309, Q342, P343, E345, Y373, H433, T437 and Q438 of said IgG Fc domain.

In one such preferred compound according to the invention or used in a method of the invention (“compound B1”), amino acid V422 and optionally one or more amino acids selected from the group consisting of R355, Q418, Q438 and P445 are replaced by another amino acid.

In yet another preferred compound according to the invention or used in a method of the invention (“compound B2”), amino acid V422 is replaced by another amino acid and the compound comprises amino acids R355, Q418 and P445.

In yet another preferred compound according to the invention or used in a method of the invention (“compound B3”), amino acids R355, Q418 and V422 are replaced by another amino acid and the compound comprises amino acid P445.

In yet another preferred compound according to the invention or used in a method of the invention (“compound B4”), amino acids R355, Q418, V422 and P445 are replaced by another amino acid.

In all these compounds B, B1, B2, B3 and B4 it is further preferred that the second epitope that is unaltered or that is altered such that RF binding to said epitope is reduced with at most 50% as compared to binding to an unaltered second RF epitope comprises amino acids R255, L309, Q342, P343, E345, Y373, H433, H435 and T437.

In a further preferred compound (“compound C”) according to, or used in accordance with the invention, the compound comprises amino acid H435 and optionally H433 and/or T437 and amino acids V422, one or more amino acids selected from the group consisting of R255, L309, Q342, P343, E345 and Y373 and optionally one or more amino acids selected from the group consisting of R355, Q418, Q438 and P445 are replaced by another amino acid.

In one such preferred compound according to the invention or used in a method of the invention (“compound C1”) amino acids R255, L309, Q342, P343, E345, Y373, R355 Q418, V422 and 445 are replaced by another amino acid and the compound comprises amino acids H433, H435 and T437.

In yet another preferred compound according to the invention or used in a method of the invention (“compound D”) amino acid V422, one or both amino acids selected from H433 and T437 and optionally one or more amino acids selected from the group consisting of R355, Q418, Q438 and P445 are replaced by another amino acid and the compound comprises amino acid H435 and optionally one or more amino acids selected from the group consisting of R255, L309, Q342, P343, E345 and Y373.

In one such preferred compound according to the invention or used in a method of the invention (“compound D1”) amino acids R355, Q418, V422, H433, T437 and P445 are replaced by another amino acid and the compound comprises amino acids R255, L309, Q342, P343, E345, Y373 and H435.

In one preferred compound according to the invention or used in a method of the invention (“compound E”) at least two amino acids selected from the group consisting of R255, L309, Q342, P343, E345, Y373, V422, H433, H435 and T437 are replaced by another amino acid and the compounds comprises one or more amino acids selected from the group consisting of R255, L309, Q342, P343, E345, Y373, H433, H435, T437, R355, Q418, V422 and P445.

In one such compound according to the invention or used in a method of the invention (“compound E1”) amino acids H435 and/or V422 and one or more amino acids selected from the group consisting of R255, L309, Q342, P343, E345, Y373, H433 and T437 are replaced by another amino acid and the compound comprises one or more amino acids selected from the group consisting of R255, L309, Q342, P343, E345, Y373, H433, H435, T437, R355, Q418, V422 and P445.

In yet another preferred compound according to the invention or used in a method of the invention (“compound E2”) amino acids H435 and V422 and optionally one or more amino acids selected from the group consisting of R255, L309, Q342, P343, E345, Y373, H433 and T437 are replaced by another amino acid and the compound comprises one or more amino acids selected from the group consisting of R255, L309, Q342, P343, E345, Y373, H433, T437, R355, Q418 and P445.

In yet another preferred compound according to the invention or used in a method of the invention (“compound E3”) amino acids H435 and V422, and optionally amino acid Q438, are replaced by another amino acid and the compound comprises amino acids R255, L309, Q342, P343, E345, Y373, H433, T437, R355, Q418 and P445.

In yet another preferred compound according to the invention or used in a method of the invention (“compound E4”) amino acids H435, V422, R255 and L309 are replaced by another amino acid and the compound comprises amino acids Q342, P343, E345, Y373, H433, T437, R355, Q418 and P445.

In yet another preferred compound according to the invention or used in a method of the invention (“compound E5”) amino acids H435, V422, Q342, P343, E345 and Y373 are replaced by another amino acid and the compound comprises amino acids R255, L309, H433, T437, R355, Q418 and P445.

In yet another preferred compound according to the invention or used in a method of the invention (“compound E6”) amino acids H435, V422, H433 and T437 are replaced by another amino acid and the compound comprises amino acids R255, L309, Q342, P343, E345, Y373, R355, Q418 and P445.

In yet another preferred compound according to the invention or used in a method of the invention (“compound E7”) amino acids H435, V422, R355, Q418 and P445 are replaced by another amino acid and the compound comprises amino acids R255, L309, Q342, P343, E345, Y373, H433 and T437.

In a compound according to the invention or used in accordance with the invention optionally amino acid Q438 is further replaced by another amino acid. This mutation may be present in any compound as described herein Amino acid Q438 may be part of the first epitope that is altered or of the second epitope that is unaltered or that is altered such that RF binding to said epitope is reduced with at most 50% as compared to binding to an unaltered second RF epitope, but cannot be part of both the first and second RF epitope in the same compound. Hence, for all compounds described herein according to the invention or used in accordance with the invention, the proviso applies that either none of the RF epitopes or either the first RF epitope or the second RF epitope comprises amino acid Q438, but not both RF epitopes. Further, if amino acid Q438 is replaced by another amino acid in the first epitope that is altered, the second epitope that is unaltered or that is altered such that RF binding to said epitope is reduced with at most 50% as compared to binding to an unaltered second RF epitope does not comprise Q438.

Also provided is a compound comprising a recombinant human IgG class fragment crystallizable (Fc) domain, wherein in said Fc domain amino acids H435 and V422, and one or more amino acids selected from the group consisting of H433, T437 and Q438 are replaced by another amino acid. Preferably, amino acids H433, H435, T437 and V422 are replaced by another amino acid, and optionally amino acid Q438 is replaced by another amino acid. More preferably, amino acids H433, H435, T437, Q438 and V422 are replaced by another amino acid, and at least one amino acid selected from the group consisting of R255, L309, Q342, P343, E345, R355, Y373, Q418, P445 is replaced by another amino acid. More preferably, amino acids R255, L309, Q342, P343, E345, R355, Y373, Q418, V422, H433, H435, T437 and P445 are replaced by another amino acid, and optionally amino acid Q438 is replaced by another amino acid. In this compound amino acids in both a first and second RF epitope as defined herein are altered. Said IgG class Fc domain is preferably a IgG1, IgG2 or IgG4 class Fc domain, more preferably a IgG1 class Fc domain.

Such compound is further advantageously used in a method according to the invention, whereby said method comprises contacting the sample of the subject with said compound and determining binding of RF to said compound, for instance as a negative control and/or to determine residual binding activity to the compound with altered first and second RF epitopes. Also provided is a compound comprising a recombinant human IgG class fragment crystallizable (Fc) domain, wherein in said Fc domain R255, Y278, R292, E293, L309, Q342, P343, E345, R355, Y373, Q418, V422, H433, H435, T437 and P445 are replaced by another amino acid. In this compound amino acids in a first, second and third RF epitope as defined herein are altered. Optionally, amino acid Q438 is further replaced by another amino acid. Said IgG class Fc domain is preferably a IgG1, IgG2 or IgG4 class Fc domain, more preferably a IgG1 class Fc domain.

In these compounds, the amino acids sequence of the compounds other than at positions R255, Y278, R292, E293, L309, Q342, P343, E345, R355, Y373, Q418, V422, H433, H435, T437 and/or P445 and optionally Q438 is further preferably at least 80% identical to the sequence present in a naturally occurring human IgG molecule, more preferably at least 85%, more preferably at least 90%, more preferably at least 95%, more preferably at least 98%, more preferably at least 99%, most preferably substantially identical to or identical to a sequence present in a naturally occurring human IgG molecule. Such compound is further advantageously used in a method according to the invention, whereby said method comprises contacting the sample of the subject with said compound and determining binding of RF to said compound.

Further provided is a compound comprising a recombinant human IgG class fragment crystallizable (Fc) domain, wherein in said Fc domain amino acid Q438 is replaced by another amino acid. Said IgG class Fc domain is preferably a IgG1, IgG2 or IgG4 class Fc domain, more preferably a IgG1 class Fc domain. In this compound, the amino acids sequence of the compound other than Q438 is preferably at least 80% identical to the sequence present in a naturally occurring human IgG molecule, more preferably at least 85%, more preferably at least 90%, more preferably at least 95%, more preferably at least 98%, more preferably at least 99%, most preferably substantially identical to or identical to a sequence present in a naturally occurring human IgG molecule. In particular, amino acids R255, Y278, R292, E293, L309, Q342, P343, E345, R355, Y373, Q418, V422, H433, H435, T437 and P445 are preferably maintained, i.e. not replaced by another amino acid. Such compound is advantageously used in a method for typing rheumatoid factor (RF), for determining a rheumatoid factor (RF) reactivity profile characteristic for a disease or condition or for determining a treatment strategy for a subject as described herein, whereby said method comprises contacting the sample of the subject with said compound and determining binding of RF to said compound. Alternatively, such compound is advantageously combined with another compound or combination according to the invention and used in a method according to the invention, whereby said method comprises contacting the sample of the subject with said compound and determining binding of RF to said compound.

Also provided is a compound comprising a recombinant human IgG class fragment crystallizable (Fc) domain, wherein in said Fc domain amino acids H435, Q418, R355 and P445 are replaced by another amino acid and the compound comprises amino acids H433, T437, Q438 and V422. Such compound is advantageously used in a method for typing rheumatoid factor (RF), for determining a rheumatoid factor (RF) reactivity profile characteristic for a disease or condition or for determining a treatment strategy for a subject as described herein, whereby said method comprises contacting the sample of the subject with said compound and determining binding of RF to said compound. Alternatively, such compound is advantageously combined with another compound or combination according to the invention and used in a method according to the invention, whereby said method comprises contacting the sample of the subject with said compound and determining binding of RF to said compound.

Also provided is a compound comprising a recombinant human IgG class fragment crystallizable (Fc) domain, wherein in said Fc domain one or more amino acids selected from the group consisting of H433, T437, Q438 and V422 are replaced by another amino acid and the compound comprises two or more amino acids selected from the group consisting of H435, R255 and L309, preferably the compound comprises amino acids H435, R255 and L309. In a preferred embodiment, amino acids H433, T437, Q438 and V422 are replaced by another amino acid and the compound comprises amino acids H435, R255 and L309. Such compound is advantageously used in a method for typing rheumatoid factor (RF), for determining a rheumatoid factor (RF) reactivity profile characteristic for a disease or condition or for determining a treatment strategy for a subject as described herein, whereby said method comprises contacting the sample of the subject with said compound and determining binding of RF to said compound.

Alternatively, such compound is advantageously combined with another compound or combination according to the invention and used in a method according to the invention, whereby said method comprises contacting the sample of the subject with said compound and determining binding of RF to said compound.

Also provided is a compound comprising a recombinant human IgG class fragment crystallizable (Fc) domain, wherein in said Fc domain amino acids H433, T437, Q438 and V422 are replaced by another amino acid and the compound comprises amino acids H435, Q418, R355 and P445. Such compound is advantageously used in a method for typing rheumatoid factor (RF), for determining a rheumatoid factor (RF) reactivity profile characteristic for a disease or condition or for determining a treatment strategy for a subject as described herein, whereby said method comprises contacting the sample of the subject with said compound and determining binding of RF to said compound. Alternatively, such compound is advantageously combined with another compound or combination according to the invention and used in a method according to the invention, whereby said method comprises contacting the sample of the subject with said compound and determining binding of RF to said compound.

Also provided is a compound comprising a recombinant human IgG class fragment crystallizable (Fc) domain, wherein in said Fc domain amino acids Q418, R355 and P445 are replaced by another amino acid and the compound comprises amino acid V422 and two or more amino acids selected from the group consisting of R255, L309, Q342, P343, E345, Y373, H433, H435 and T437. In a preferred embodiment, the compound comprises amino acids V422 and H435 and one or more amino acids selected from the group consisting of R255, L309, Q342, P343, E345, Y373, H433 and T437, more preferably the compound comprises V422, R255, L309, Q342, P343, E345, Y373, H433, H435 and T437. Such compound is advantageously used in a method for typing rheumatoid factor (RF), for determining a rheumatoid factor (RF) reactivity profile characteristic for a disease or condition or for determining a treatment strategy for a subject as described herein, whereby said method comprises contacting the sample of the subject with said compound and determining binding of RF to said compound. Alternatively, such compound is advantageously combined with another compound or combination according to the invention and used in a method according to the invention, whereby said method comprises contacting the sample of the subject with said compound and determining binding of RF to said compound.

Also provided is a compound comprising a recombinant human IgG class fragment crystallizable (Fc) domain, wherein in said Fc domain amino acids V422 and two or more amino acids selected from the group consisting of R255, L309, Q342, P343, E345, Y373, H433, H435 and T437 are replaced by another amino acid and the compound comprises amino acids Q418, R355 and P445. In a preferred embodiment, amino acids V422 and H435 and optionally one or more amino acids selected from the group consisting of R255, L309, Q342, P343, E345, Y373, H433, and T437 are replaced by another amino acid and the compound comprises amino acids Q418, R355 and P445. In a further preferred embodiment, amino acids V422, H433, H435 and T437 and optionally one or more amino acids selected from the group consisting of R255, L309, Q342, P343, E345 and Y373 are replaced by another amino acid and the compound comprises amino acids Q418, R355 and P445. In a further preferred embodiment, amino acids V422, R255, L309, Q342, P343, E345, Y373, H433, H435 and T437 are replaced by another amino acid and the compound comprises amino acids Q418, R355 and P445. Such compound is advantageously used in a method for typing rheumatoid factor (RF), for determining a rheumatoid factor (RF) reactivity profile characteristic for a disease or condition or for determining a treatment strategy for a subject as described herein, whereby said method comprises contacting the sample of the subject with said compound and determining binding of RF to said compound. Alternatively, such compound is advantageously combined with another compound or combination according to the invention and used in a method according to the invention, whereby said method comprises contacting the sample of the subject with said compound and determining binding of RF to said compound.

The compounds and preferred compounds indicated above are provided as such and can be used alone or in combination with 1 or more of such compound in a method of the invention, such as in combination with 1, 2, 3, 4, 5, 6, 7, etc of such compounds. Similarly, these compounds and preferred compounds are present alone or in combination with 1 or more of such compounds in a combination according to the invention, such as in combination with 1, 2, 3, 4, 5, 6, 7, etc of such compounds. It is preferred that at least one of the compounds A, A1, A2, A3, A4, A5, A6, B, B1, B2, B3, B4, C, C1, D and D1 are used in a method of the invention.

More preferably at least two of such compounds are used in a method of the invention. It is further preferred that at least one of compounds A, A1, A2, A3, A4, A5 and A6 and one of compounds B, B1, B2, B3 and B4 is used, more preferably more preferably one of compound A1, A2, A3, A4, A5 and A6 and one of compounds B1, B2, B3 and B4 is used. These compounds can be combined with any other compound A, A1, A2, A3, A4, A5, A6, B, B1, B2, B3, B4, C, C1, D, D1, E, E1, E2, E3, E4, E5, E6, E7 and combinations thereof.

In a preferred embodiment, the invention provides a method for typing rheumatoid factor (RF), comprising:
contacting a sample from a subject, said sample comprising or suspected of comprising RF, with at least two compounds comprising a recombinant human

IgG1, IgG2 or IgG4, preferably IgG1, class fragment crystallizable (Fc) domain,
wherein said Fc domain of a first compound A comprises:

1) a first RF epitope comprising amino acids at positions 433, 435 and 437 and optionally amino acids at positions 255, 309, 342, 343, 345 and/or 373 of said Fc domain, whereby said first RF epitope is altered such that RF binding to said epitope is reduced with at least 20% as compared to binding to an unaltered first RF epitope and

2) a second RF epitope comprising amino acids at position 355, 418 and 422 and optionally at position 445 of said Fc domain, whereby said second RF epitope is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, whereby in said compound A:
amino acids H433, H435 and T437 are replaced by another amino acid and the compound comprises amino acids R255, L309, Q342, P343, E345 and Y373, or
amino acids R255, L309 and H435, are replaced by another amino acid and the compound comprises amino acids Q342, P343, E345, Y373, H433, and T437, or
amino acids Q342, P343, E345, Y373 and H435 are replaced by another amino acid and the compound comprises amino acids R255, L309, H433 and T437, or

amino acids R255, L309, H433, H435 and T437 are replaced by another amino acid and the compound comprises amino acids Q342, P343, E345 and Y373, or
amino acids R255, L309, Q342, P343, E345, Y373, H433, H435 and T437 are replaced by another amino acid

wherein said Fc domain of a second compound B comprises:

1) a first RF epitope comprising amino acids at position 355, 418 and 422 and optionally at position 445 of said Fc domain, whereby said first RF epitope is altered such that RF binding to said epitope is reduced with at least 20% as compared to binding to an unaltered first RF epitope and

2) a second RF epitope comprising amino acids at positions 433, 435 and 437 and optionally amino acids at positions 255, 309, 342, 343, 345 and/or 373 of said Fc domain, whereby said second RF epitope is unaltered or that is altered such that RF binding to said epitope is reduced with at most 50% as compared to binding to an unaltered second RF epitope, with the proviso that the percentage of reduction in binding to said first RF epitope is higher than the percentage of reduction in binding to said second RF epitope, whereby in said compound B:
amino acid V422 is replaced by another amino acid and the compound comprises amino acids R355, Q418 and P445, or amino acids R355, Q418 and V422 are replaced by another amino acid and the compound comprises amino acid P445, or
amino acids R355, Q418, V422 and P445 are replaced by another amino acid, and
determining the percentage of RF that binds to each of said at least two compounds as compared to binding to a compound comprising an Fc domain wherein said first and said second RF epitope are both unaltered and that is preferably otherwise identical to said at least two compounds.

Replacement of an amino acid at certain position by another amino acid means that the amino acid as it is present in human IgG, preferably human IgG1, IgG2 or IgG4, more preferably IgG1 or IgG2, is replaced by any other amino acid, as long as the requirement is fulfilled that the first RF epitope is altered such that RF binding to said epitope is reduced with at least 20% as compared to binding to an unaltered first RF epitope and the second epitope is unaltered or altered such that RF binding to said epitope is reduced with at most 50% as compared to binding to an unaltered second RF epitope. The amino acid can be replaced by any other amino acid. A skilled person is well capable of determining particularly suitable amino acid substitutions, for instance by preparing altered RF epitopes and determining binding of RF to the epitope with a method as described herein. In one preferred embodiment, the amino acids are replaced by the amino acid that is present at the same position in mouse IgG2b. However, as demonstrated in the examples for several om the amino acid residues, replacement by any of the tested amino acids results in a sufficient reduction of binding to the relevant epitope by RF that does bind to the unaltered epitope. In particular, it is preferred that in a compound according to the invention, present in a combination according to the invention or used in a method of the invention:
if R253 is replaced, it is preferably replaced by an amino acid selected from the group consisting of L, A V, I and M., most preferably by L,
if L309 is replaced, it is preferably replaced by an amino acid selected from the group consisting of Q, N, T and S, most preferably by Q,
if Q342 is replaced, it is preferably replaced by an amino acid selected from the group consisting of L, A V, I and M, most preferably by L,
if P343 is replaced, it is preferably replaced by an amino acid selected from the group consisting of L, A V, I and M, most preferably by V,
if E345 is replaced, it is preferably replaced by an amino acid selected from the group consisting of L, A V, I and M, most preferably by A,
if Y373 is replaced, it is preferably replaced by an amino acid selected from the group consisting of S, T, N and Q, most preferably by N,
if H433 is replaced, it is preferably replaced by an amino acid selected from the group consisting of H, K and R, most preferably by K,
if H435 is replaced, it is preferably replaced by an amino acid selected from the group consisting of F, W and Y, most preferably by Y,
if T437 is replaced, it is preferably replaced by an amino acid selected from the group consisting of L, A V, I and M, most preferably by L,
if Q438 is replaced, it is preferably replaced by an amino acid selected from the group consisting of H, K and R, most preferably by K, if R355 is replaced, it is preferably replaced by an amino acid selected from the group consisting of L, A V, I and M, most preferably by A,
if Q418 is replaced, it is preferably replaced by an amino acid selected from the group consisting of D and E, most preferably by E, if V422 is replaced, it is preferably replaced by an amino acid selected from the group consisting of S, T, N and Q, most preferably by S,
if P445 is replaced, it is preferably replaced by an amino acid selected from the group consisting of L, A V, I and M, most preferably by L.

A compound according to the invention or used in a method according to the invention preferably comprises a recombinant human IgG1, IgG2 or IgG4 class Fc domain, preferably a recombinant human IgG1 class Fc domain. As used herein, a “recombinant human IgG1, IgG2 or IgG4 class Fc domain” means that the amino acids sequence of the Fc domain, other than the sequences of the first, second and optionally third RF epitopes as defined herein, is at least 80% identical to the corresponding sequence of a naturally occurring human IgG1, IgG2 or IgG4 molecule, respectively. Preferably the amino acids sequence of the Fc domain, other than the sequences of the first, second and optionally third RF epitopes as defined herein, is at least 85%, more preferably at least 90%, more preferably at least 95%, more preferably at least 98%, more preferably at least 99% identical to the corresponding sequence of a naturally occurring human IgG1, IgG2 or IgG4 class molecule, respectively. I.e. the sequence of said human IgG1, IgG2 or IgG4 class Fc domain is at least 85%, more preferably at least 90%, more preferably at least 95%, more preferably at least 98%, more preferably at least 99% identical to the sequence of a human IgG1, IgG2 or IgG4 class molecule, respectively, with the exception of the mutations defined herein, in particular alterations in the first and optionally second and/or third RF epitopes as defined herein. In a particular embodiment, the sequence of said human IgG1, IgG2 or IgG4 class Fc domain is essentially identical or identical to the sequence of a human IgG1, IgG2 or IgG4 class molecule, respectively, with the exception of the mutations defined herein, in particular alterations in the first and optionally second and/or third RF epitopes as defined herein.

The step of contacting a sample from the subject with the at least one or at least two compounds is preferably in aqueous solution. A method according to the invention further comprises a step of determining binding of RF to the at least one or at least two compounds. Said determining preferably comprises detecting binding of RF to said compound(s) and optionally quantifying said binding. Several methods are known in the art to detect binding of an antibody (in this case the RFs) to its antigen (in this case the compound comprising a recombinant IgG class Fc domain). One example is the use of enzyme-linked immunosorbent assays (ELISAs), for which a suitable method is detailed in the examples herein. In brief, the compound comprising a recombinant human IgG class fragment crystallizable (Fc) domain is immobilized on flat-bottom plates, after which serum samples comprising or suspected of comprising RFs are added to the wells. After washing, anti-RF antibodies, such as labeled anti-IgM-RF can be added to detect bound RF.

Quantifying binding is for instance relative quantification and can performed by comparing binding of RF in a sample to a compound comprising a recombinant human IgG class Fc domain as described with binding of RF from the same sample to a reference compound. Said reference compound is for instance a human IgG class Fc domain with an unaltered first RF epitope and an unaltered second RF epitope. The remainder or the amino acid residues in the reference compound are preferably identical to the remainder of the amino acid residues in the at least one or at least two compounds used in a method of the invention. This way, the reduction in binding of the same population or RFs to a compound used in accordance with the invention and a naturally occurring IgG molecule can be determined for each compound.

In a further preferred embodiment, determining binding of RF to the at least one compound or the at least two compounds comprises determining the percentage of RF that binds to said at least one compound or to each of said at least two compounds as compared to binding to a compound comprising an Fc domain wherein said first and said second RF epitope are both unaltered, and that is preferably otherwise identical to said at least one or at least two compounds. If for each compound binding of RF thereto is determined, ratio's of specific RFs with specific reactivity present in the sample can be calculated. Hence, determining the percentage of binding encompasses determining the ratio of binding of RF to a compound comprising a recombinant human IgG class Fc domain according to the invention and binding to a reference compound and determining the ratio of binding of RF to different compounds comprising a recombinant human IgG class Fc domain according to the invention.

Many different compounds can be used in a method of the invention, whereby said method comprises contacting the sample with each of said compounds and determining for each of said compounds binding of RF to the compound. For instance two compounds can be used wherein in a first compound all amino acids of the entire first epitope comprising amino acids R255, L309, Q342, P343, E345, Y373, H433, H435 and T437 are replaced by another amino acid and additional compounds are used wherein only subsets of amino acids within this epitope are replaced by another amino acid and the other amino acids are maintained to further dissect binding of a specific sample or population of RFs to this epitope. Examples of such compounds are compounds A1 to A6 described herein above. This allows determining the amino acids within a single epitope that contribute most to the reactivity of a particular RF or population of RFs against the epitope.

As used herein “typing rheumatoid factor” refers to determining the part or parts of the Fc domain which a particular RF or population of RFs recognizes, i.e. to determining the epitope and/or most important amino acids therein of a particular RF or population of RFs. Typing RF is thus preferably typing RF on the basis of binding thereof to at least one RF epitope. A “RF reactivity profile” as used herein refers to a profile of RFs in terms of the epitope or epitopes and/or the amino acids thereof that are bound by RFs in a sample of a subject or that is characteristic of a particular disease or condition. A “population of RFs” refers to multiple RFs, typically the RFs present in the circulation or other body fluid of a particular subject or patient. Typically each RF, i.e. each autoantibody, recognizes a specific epitope. It is possible that in a population of RFs all RFs recognize the same epitope. Typically, however, a population of RFs is a heterogenous population, meaning that it contains different RFs that recognize different epitopes. In addition, within a single epitope the amino acids that contribute most to binding of a particular RF may be different. Differences of such heterogenous populations of RFs, such as RFs isolated from different subjects or patients, are typically characterized by having different ratio's of RF's recognizing different epitopes. As shown in the Examples herein, ratio's of RFs recognizing different epitopes or the absence of a specific RF recognizing one particular epitope in a sample can be clinically relevant.

The methods disclosed herein allow the characterization of RFs in a sample of a subject. Hence, in one embodiment, typing RF is characterizing RF. As such “typing RF” preferably comprises determining the epitope that is or the amino acids that are recognized by RF or the epitope or epitopes or amino acids that are recognized by a population of RFs.

Using samples of patients that have been diagnosed with a particular disease or condition it is possible to use the methods of the invention to determine a RF reactivity profile that is characteristic of the particular disease or condition. This is for instance done by comparing the specific reactivity of the RFs present in the sample (e.g. the epitopes that are bound by RFs in the sample and the ratio's between different RFs) with the reactivity of RFs in samples from healthy subjects or from subjects suffering from other conditions or diseases. Hence, in one embodiment the invention provides a method for determining a RF reactivity profile characteristic for a disease or condition, preferably a disease or condition characterized by the presence of rheumatoid factor, comprising typing RFs in one or more samples, preferably a plurality of sample from patients suffering from the disease or condition with a method for typing RF according to the invention. Said method preferably comprises contacting said sample with at least two of said compounds comprising a recombinant human IgG class Fc domain, wherein said second RF epitope in each of said two compounds is different, the method further comprises determining binding of RF to said at least two compounds. Said method further preferably comprises determining the percentage of RF that binds to the at least one compound or each of the at least two compounds as compared to binding to a compound comprising an Fc domain wherein said first and said second RF epitope are both unaltered, and that is preferably otherwise identical to said at least one or two compounds, and optionally determining ratio's of RFs binding to different compounds. Preferably said percentages and/or ratio's are compared with the percentages and/or ratio's of binding of RFs binding in a reference sample (e.g. healthy subjects or subjects suffering from another disease) to the same at least one compound or at least two compounds. Based thereon, a profile can be established for the particular disease or condition.

As described in the examples herein, profiles have been already been established, including for the risk of progression to arthritis. Such profile as described in the Examples or established using a method of the invention can be used as diagnosis of a specific disease or condition characterized by the presence of RF. Hence, in one embodiment, a method of the invention for typing RF comprises establishing a RF reactivity profile based on said typing or RF. In another embodiment, a method of the invention for typing RF comprises diagnosis. In another embodiment, a method of the invention for typing RF is part of diagnosis. Also provided is a use of a compound or combination of a first compound A and a second compound B according to the invention in diagnosis. Said diagnosis preferably comprises a step of typing rheumatoid factor with a method according to the invention. Said diagnosis may further comprise further methods or assays, such as commercially available RF assays.

“Diagnosis” as used herein in a method or use according to the invention is preferably diagnosis of a specific disease or condition characterized by the presence of RF, such as rheumatoid arthritis (RA), arthralgia, systemic lupus erythematosus (SLE), Sjögren syndrome, interstitial pulmonary fibrosis, hepatitis B, chronic liver disease, and chronic hepatitis, essential mixed cryoglobulinemia, primary biliary cirrhosis, infectious mononucleosis and any chronic viral infection, bacterial endocarditis, leprosy, sarcoidosis, tuberculosis, syphilis, visceral leishmaniasis, malaria, leukemia, dermatomyositis and systemic sclerosis. In a preferred embodiment said disease or condition is selected from the groups consisting of RA, arthralgia and Sjögren syndrome.

The examples herein show that generally speaking, a broader RF response appears to signal a more pathogenic RF response. The data show that significant skewing of an RF response towards either the Tail or the ER epitopes is associated with a lower risk of arthritis development in arthralgia patients and less anti-citrullinated protein antibody (ACPA) positivity. Furthermore, RF responses in pSS patients seem to lack reactivity against Tail altogether.

In one embodiment is provided a method for typing RF according to the invention wherein a ratio of the level of RF that binds to compound A as compared to the level of RF that binds to compound B is determined and the more said ratio deviates from 1, the lower the risk of progression to arthritis, preferably rheumatoid arthritis. Preferably said risk of progression is risk of progression from arthralgia to arthritis, preferably rheumatoid arthritis.

A ratio of the level of RF that binds to compound A as compared to the level of RF that binds to compound B can be determined using any suitable method. For instance, a ratio of the percentage of RF that binds to compound A as compared to binding to a compound comprising an Fc domain wherein said first and said second epitope are both unaltered to the percentage of RF that binds to compound B as compared to binding to a compound comprising an Fc domain wherein said first and said second epitope are both unaltered is determined.

The more a ratio of the level of RF that binds to compound A as compared to the level of RF that binds to compound B deviates from 1 the lower the risk of progression to arthritis. “Deviate” as used herein means either lower or higher than 1. The more the ratio deviates from 1, i.e. the higher or lower than 1, the lower the risk of progression to arthritis. Preferably, a ratio of higher than 2 or lower than 0.5 is indicative of a low risk of progression to arthritis, such as a ratio higher than 3, or higher than 4 and a ratio lower than 0.33 or lower than 0.25. Progression to arthritis, preferably rheumatoid arthritis, is preferably progression from arthralgia to arthritis. As used herein “arthralgia” refers to joint pain, independent of the cause, which may for instance be injury, infection, illness, or an allergic reaction. An individual suffering from arthralgia may be at risk of developing RA. Hence, in such method a sample is preferably a sample comprising or suspected of comprising RF that is obtained from a subject suffering from arthralgia.

The compound comprising an Fc domain wherein said first and said second epitope are both unaltered is preferably otherwise identical to said compound A.

Besides providing insight into the pathogenic potential of RF responses, the findings may also be of practical use for optimizing existing RF assays. Measuring RFs is important for diagnosing RA and predicting disease severity, but the assays currently used in the clinic are not standardized and lack specificity. In the examples it is shown that RF reactivity patterns associate with clinical outcomes in arthralgia patients. By using these novel IgG-targets, instead of the current wildtype human or rabbit IgGs, clinically non-relevant RF responses can potentially be eliminated from RF assays. For example, by using IgG-ER as the target antibody, RF responses exclusively directed against the Tail epitopes would not be detected. Since these RF responses are virtually restricted to arthralgia patients that do no develop arthritis, RF assays would gain in specificity. The gain in specificity from replacing IgG-WT with IgG-ER would not result in a lower sensitivity for diagnosing RA, since the sole patient that developed arthritis with a Tail/ER ratio >4 (FIG. 8B) had only one swollen joint and was diagnosed with undifferentiated arthritis rather than RA. IgG-ER may thus replace IgG-WT as the primary target to measure RF. Additionally measuring reactivity against IgG-Tail may have added value in IgG-ER positive patients. Arthralgia patients with very high levels (>100 AU/ml) of anti-IgG-ER RF without any anti-IgG-Tail reactivity did not develop arthritis. Currently, equal weight is given to RF and ACPA status and level in the RA classification criteria, despite the lower specificity of RF testing.

Standardizing RF assays by using better defined, more specific RF targets could improve the value of the classification criteria. Hence, also provided is a RA diagnostic assay wherein a compound according to the invention is used as binding partner for a patient sample.

If it is established that a subject is suffering or at risk of suffering from a disease or condition characterized by the presence of RF using a method for typing RF of the invention, it can be determined if and how the subject can be treated. Treatment strategies for diseases or condition characterized by the presence of RF are known in the art and can be assigned to the subject. Provided is therefore, a method for determining a treatment strategy for a subject, comprising typing RFs in a sample of the subject with a method for typing RF according to the invention, and determining a treatment strategy for said subject if said typing and/or the established RF reactivity profile indicates that said individual is suffering from or at risk of suffering from a particular disease or condition characterized by the presence of RF.

Also provided is a method of treatment of a subject in need thereof, comprising typing RFs in a sample of the subject using a method for typing RF according to the invention and providing said subject with treatment if said typing and/or the established RF reactivity profile indicates that said individual is suffering from or at risk of suffering from a particular disease or condition characterized by the presence of RF. Treatment strategies for diseases or condition characterized by the presence of RF are known in the art.

As used herein, the terms “treatment,” “treat,” and “treating” refer to reversing, alleviating, delaying the onset of, or inhibiting the progress of a disease or condition, or one or more symptoms thereof. In some embodiments, treatment may be administered after one or more symptoms have developed. In other embodiments, treatment may be administered in the absence of symptoms, after typing of RF indicate that the subject is suffering from or at risk of suffering from a disease or condition characterized by the presence of RF but the subject is not yet experiencing symptoms. Treatment may also be continued after symptoms have resolved, for example to prevent or delay their recurrence.

Features may be described herein as part of the same or separate aspects or embodiments of the present invention for the purpose of clarity and a concise description. It will be appreciated by the skilled person that the scope of the invention may include embodiments having combinations of all or some of the features described herein as part of the same or separate embodiments.

The invention will be explained in more detail in the following, non-limiting examples.

### BRIEF DESCRIPTION OF THE DRAWINGS

FIG. 1: CH2-CH3 domain amino acid sequences of the IgG targets

Amino acid sequences of CH2 and CH3 domains of the engineered recombinant IgG targets are depicted, with human→mouse amino acid mutations underlined and an additional P→L mutation underlined. Numbering is EU-numbering.

FIG. 2: Development of recombinant IgG targets

A) IgM-RF reactivity against human IgG, rabbit IgG, and all four mouse IgG subclasses was tested with different dilutions of a pooled RF standard serum

(RELARES). OD: optical density.

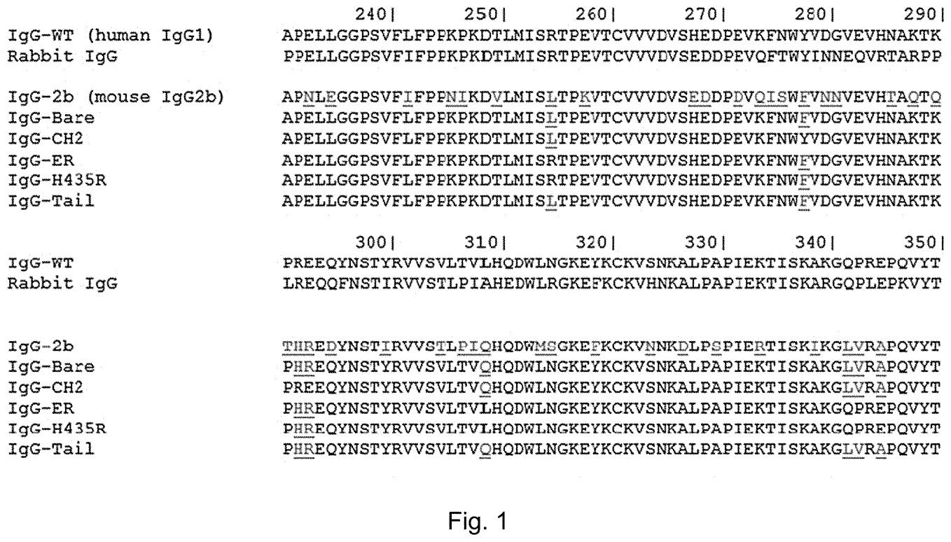

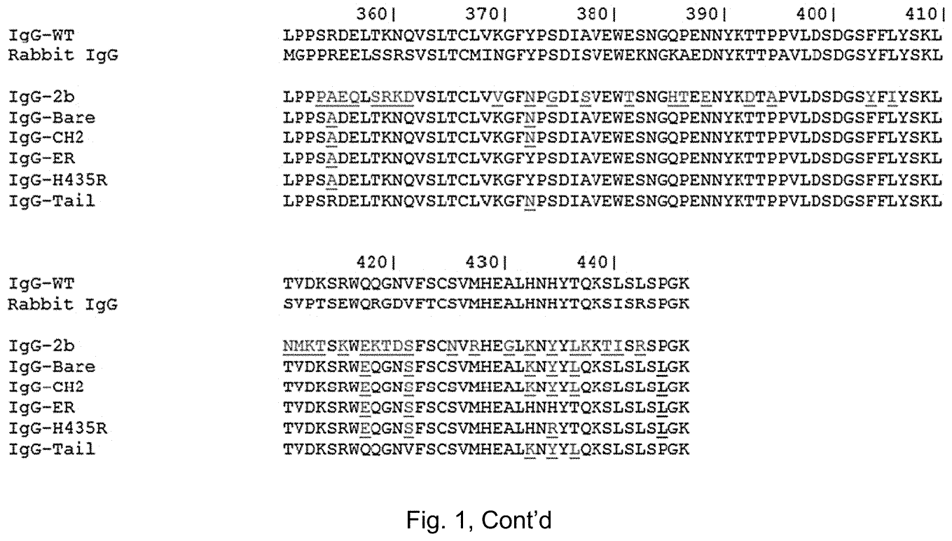

B) Front, side and back views of the ‘parent’ IgG target, designated IgG-bare, with 15 amino acid replacements in the IgG1 Fc domain where the ‘human amino acid’ was replaced with the ‘mouse IgG2b amino acid’ as well as an additional P445L mutation, all indicated in light-grey. Note that the molecule is symmetrical and that in reality the mutations are present in both chains. See FIG. 1 for amino acid sequences.

C) IgM-RF reactivity against the IgG-B are target, and control IgG-WT and mouse IgG2b (IgG-2b) targets, tested with RELARES.

D) Front views of three additional recombinant IgG targets, based on IgG-bare, with different clusters of human amino acids reintroduced, indicated in mid-grey in circles. Cluster A located in the CH2 domain (‘IgG-CH2’); cluster B at the CH2-CH3 elbow region (‘IgG-ER’); cluster C at the tip of the CH3 domain (‘IgG-Tail’).

E) Binding properties of the IgG targets analyzed using two monoclonal IgM-RFs: RF61 and RF-AN. The middle panel shows where the two monoclonal RFs interact with IgG-Fc, based on previously published crystal structures (Duquerroy et al. 2007; Corper et al. 1997), and was created with Discovery Studio 4.5 software, using structures 2J6E and 1ADQ from the RSCB Protein Data Bank (Berman et al. 2000).

FIG. 3: Complement activation by the various IgG targets at different IgG concentrations, determined by measuring C3 deposition in ELISA.

FIG. 4: Reactivity against IgG targets in seropositive arthralgia patients A) Levels of IgM-RF reactivity against six recombinant IgG targets, measured in 639 seropositive (ACPA and/or RF) arthralgia patients. The Fc domains depicted_below the X-axis show the area's with mouse IgG2b amino acids in light grey. Bars show medians and interquartile range. Values below the cut-off of 1 AU/mL are depicted at 0.5 AU/mL.

B) The left X-Y plot shows the tight correlation between levels of anti-IgG-CH2 reactivity and anti-IgG-Bare reactivity. Right panel: comparison of RF levels against IgG-Tail and IgG-ER. While many samples show reactivity against both targets, some specifically react with either Tail or ER.

C) Design of the recombinant IgG target ‘IgG-H435R’, which is identical to IgG-ER, except for a histidine→arginine mutation at position 435.

D) Comparison of RF reactivity against targets IgG-ER and IgG-H435R in the seropositive arthralgia patients. (p<0.0001; Wilcoxon matched-pairs signed rank test)

FIG. 5: X-Y plots showing correlations between RF levels in arthralgia patients against various engineered recombinant IgG targets. r=Spearman's r.

FIG. 6: A) Correlations between RF reactivity against IgG-targets IgG-WT, IgG-ER and IgG-Tail and anti-CCP (aCCP) levels in the arthralgia cohort.

B) X-Y plots comparing RF reactivity against IgG-ER and IgG-Tail for 32 RF+ healthy donors (left) and 97 RA patients.

C) The degree to which reactivity against IgG-ER depends on the presence of a histidine at position 435 is compared for ACPA positive and ACPA negative arthralgia patients by plotting the H435R/ER reactivity ratio against RF reactivity towards IgG-ER for patients with at least 1 AU/ml of anti-IgG-WT RF. An RF reactivity pattern where H435 is crucial for RF reactivity against the ER region (H435R/ER ratio ≤0.1) is associated with less ACPA positivity (27% versus 63%).

D) Showing the same analysis of RF reactivity patterns as in A, but with development of arthritis within 2 years as the outcome. Data were analyzed for 465 patients with an RF level against IgG-WT ≥1 and a follow-up of two years.

FIG. 7: RF reactivity patterns and clinical outcomes in arthralgia patients

A) The degree of skewing of the RF reactivity pattern towards either the IgG-Tail or the IgG-ER target is compared for ACPA positive and ACPA negative arthralgia patients by plotting the Tail/ER reactivity ratio against RF reactivity towards IgG-WT. Comparing patients with at least 1 AU/ml of anti-IgG-WT RF (n=567, shaded area), there is 43% ACPA positivity in the group with an RF reactivity pattern skewed towards Tail (Tail/ER ratio >2) and 38% in those with RF reactivity skewed toward ER (Tail/ER ratio ≤0.1) compared to 70% in the rest of the cohort. B) Showing the same analysis of RF reactivity patterns as in A, but with development of arthritis within 2 years as the outcome. Data were analyzed for 465 patients with an RF level against IgG-WT ≥1 and a follow-up of two years.

C, D) ROC analyses showing the association between autoantibody status and development of arthritis within 2 year. ROC analysis of RF reactivity against IgG-WT yields an AUC of 0.603. Using ER, an AUC of 0.646 is obtained. Combining ER titer and a normalized Tail/ER ratio (TE) results in an AUC of 0.697. Anti-CCP on its own yields an AUC of 0.718 (p<0.0001) (D). Combination of a-CCP with WT results in enhanced association with arthritis development (AUC 0.732); combination of a-CCP with ER and TE results in a further enhanced association (AUC 0.753).

FIG. 8: A. IgG Fc structure with in dark grey all ‘bare’ positions; in middle grey additional positions screened for RF reactivity. B. Reactivity of the Relares multi-patient RF serum pool to different Ig targets.

FIG. 9: Reactivity in sera of patients with established RA to WT, ER, and ER_422 targets.

FIG. 10: A. IgG Fc structure with different ER clusters indicated. B. Reactivities in sera of patients with established RA to targets based on the ‘bare’ target, with subsets of the ER cluster of mutations reverted to human.

FIG. 11: Impact of mouse and non-mouse/non-human mutations on binding of RF. >75; >50; >20 indicates the percentage of RF binding as compared to binding to the compound with human amino acid at the relevant position.

FIG. 12: Impact of position 438 mutation in binding of RF in sera of rheumatoid arthritis patients. The graph shows relative reactivity against RF. The IgG structures show the relevant position.

FIG. 13: Impact of positions 422 and 438 mutation in binding of RF in pooled sera of rheumatoid arthritis patients. The graph shows relative reactivity against RF. The IgG structures show the relevant position.

FIG. 14: Impact of positions 433, 435, 437, and 438 in binding of RF in pooled sera of rheumatoid arthritis patients. The graph shows relative reactivity against RF. The IgG structures show the relevant position.

FIG. 15: Impact of 342, 343, 345, 373 in binding of RF in pooled sera of rheumatoid arthritis patients. The graph shows relative reactivity against RF. The IgG structures show the relevant position.

### EXAMPLES

### Materials and Methods

Production of Recombinant IgG Targets

To characterize RF binding to predicted IgG epitopes, seven different IgG molecules were produced to use as targets in RF assays. Six different human IgG1-based constant heavy chain (CH) constructs were designed: one coding for the human wildtype amino acid sequence of IgG1-CH (IgG-WT) and five with nucleotide mutations resulting in replacement of pre-determined human IgG1-CH amino acid sequences with their mouse IgG2b-CH analogs (see FIG. 1 for sequence details). The nucleotide replacements were selected by comparing the structure of human and rabbit IgG, to which most RFs bind, with mouse IgG, to which almost no RFs bind or were based on data from previous binding studies of monoclonal RFs and studies of crystal structures of monoclonal RFs in complex with IgG (Bonagura et al. 1998; Bonagura et al. 1993; Duquerroy et al. 2007; Corper et al. 1997). The mutated IgG targets were designated as follows: IgG-Bare, with 16 amino acid replacements divided over 3 clusters: one in the CH2 domain (3 amino acid replacements), one in the CH2-CH3 elbow region (9 replacements) and one at the tail end of the CH3 domain (4 replacements); IgG-CH2, with the CH2 cluster non-mutated; IgG-ER, with the elbow region cluster non-mutated; IgG-Tail, with the CH3 cluster non-mutated; IgG-H435R, identical to IgG-ER, but with an additional H435R replacement; IgG-2b, with fully mouse IgG2b CH domains (FIG. 1).

The targets were produced as full recombinant chimeric IgG antibodies, all specific for biotin, by cloning synthetic constructs coding for anti-biotin variable domains (Kohen et al. 1997; Bagci et al. 1993) into a pcDNA3.1 expression vector, together with one of the six designed IgG1-CH constructs, a mouse IgG2b-CH construct or human k constant domains. All antibodies were produced under serum-free conditions (FreeStyle 293 expression medium; Invitrogen) by co-transfecting relevant heavy-chain- and light-chain-expressing vectors in HEK 293F cells using 293fectin according to the manufacturer's instructions (Invitrogen). The cells were cultured at 37° C., 8% CO2, while shaking at 125 RPM. At day 5 of transfection the cultures were centrifuged, supernatant was harvested, filtered over a syringe filter with a pore size of 0.20 μm (Whatman Puradisc 30; Sigma-Aldrich) and loaded on a Hitrap α-kappa column (ÄKTA prime). IgGs were eluted with 0.1M glycine pH 2.5—

3. The eluate was immediately neutralized with 2M Tris HCl, pH 9, dialyzed and concentrated by multiple rounds of spinning down the sample using a 10 kDa spin column (Amicon Utra-4 Centrifugal Filter Unit) and resuspending in phosphate buffered saline (PBS). The concentration of the purified IgG was determined by measuring absorbance at 280 nm (NanoDrop 1000; Thermo Fischer Scientific) and the samples were aliquoted and stored at −20° C.

Serum Samples

Serum samples from three different patient cohorts were used. The first consisted of 639 baseline serum samples from patients included in the Reade seropositive arthralgia cohort, which has been enrolling patients with (a history of) arthralgia and a positive IgM-RF and/or IgG-ACPA test since 2004. These patients did not have arthritis at the time of first physical examination and had no history of being diagnosed with arthritis. Patients were followed for five years to determine arthritis development, with yearly visits to the clinic and extra visits in case of suspected arthritis. Presence of at least one swollen joint on physical examination of 44 joints by a trained medical doctor was defined as evidence of arthritis. The second set consisted of baseline serum samples from 97 rheumatoid arthritis (RA) patients just before starting therapy with the TNF-blocker adalimumab. The studies involving patients with arthralgia or RA were approved by the Ethics

Committee of Slotervaart Hospital and Reade, Amsterdam, The Netherlands. Written informed consent was obtained from all study participants. To obtain RF+ healthy control samples 268 sera from in-house volunteers and left-over samples from donors who were frequently boosted with tetanus toxoid were tested for RF reactivity against human IgG. 34 RF+ healthy controls were selected based on an RF reactivity level >5 AU/ml. No informed consent was obtained for the samples from the second group, because materials were leftovers from samples taken for routine diagnostic purposes.

ELISAs

RF reactivity against the individual IgG targets was analyzed in enzyme-linked immunosorbent assays (ELISAs). All target antibodies were diluted in PBS to 1 μg/ml and coated overnight at 4° C. on Nunc MaxiSorp 96-wells flat-bottom plates (Thermo Fisher Scientific). Plates were washed 5× with 0.02% Tween 20-PBS and one hundred microliters of serum samples, controls, or reference serum diluted in 0.1% Tween 20-PBS was added to the wells and incubated for 60 min, shaking, at room temperature. After washing, IgM-RF was detected by incubating the wells for 30 min with 100 μL horseradish peroxidase (HRP)-conjugated mouse monoclonal anti-human IgM (μ-chain-specific) antibodies diluted 1:1500 (0.5 mg/mL, MH-15; Sanquin) and visualized with 3,3′,5,5′-tetramethylbenzidine (100 μg/mL) in 0.11 M acetate buffer, pH 5.5, containing 0.003% H₂O₂ (Merck). The reaction was stopped with 2 M H2SO4, and optical density (OD) was read at 450 nm and 540 nm for background correction using a BioTek microtiter plate reader. Levels of IgM-RF were calculated using a calibrator curve of a national reference serum normally used in the standard IgM-RF ELISA (‘RELARES’). This reference serum has a defined IgM-RF level of 200 IU/ml (Klein and Janssens 1987). We arbitrarily defined the reference serum as containing 200 arbitrary units (AU)/ml of anti-IgG1-reactive IgM-RF and calculated the levels of reactivity against the IgG mutant targets on the linear part of the anti-IgG1 WT reactivity curve of the reference serum diluted 1:6400-1:409,600 in 2-fold dilution steps. To arrive at a cut-off value for the different assays, we analyzed a panel of 31 randomly selected healthy individuals. The median signals for 2b, Bare, and WT were 0.40 (IQR 0.25-0.55), 0.38 (IQR 0.30-0.59), and 0.46 (IQR 0.33-1.34) AU/mL, respectively. For both 2b and Bare, but not for WT, signals were log-normally distributed, and no correlation was found between 2b and Bare. Based on these results, we chose a conservatively low cut-point as mean +2 SD of the log-transformed signals for the 2b target for all targets, i.e. 1.14, which was rounded to 1 AU/mL. This results in 1/31, 3/31, and 10/31 positive samples for 2b, Bare, and WT, respectively.

Additional Target Antibodies and Monoclonal RFs

Polyclonal human IgG was obtained from Intravenous immunoglobulin (IVIG, Nanogam, Sanquin). Polyclonal rabbit IgG was purified from rabbit plasma using protein G affinity chromatography (HiTrap Prot G HP; GE Healthcare Life Sciences). Purified wildtype mouse subclass IgG antibodies were purchased from BD Biosciences.

Two monoclonal IgM-RFs, RF61 and RF-AN, were produced as described previously (Falkenburg et al. 2017).

Statistical Analysis

Logistic regression was carried out using R v3.4.3, using log-transformed values of antibody levels as continuous input variables, and development of arthritis within 2 years as categorical response variable. For some analyses, the ‘skewedness’ towards mostly either ER or Tail reactivity was used as additional input variable, expressed as a ‘normalized ratio’: TE=if else (Tail/ER<1, 0.2/(Tail/ER), Tail/ER); the factor 0.2 accounts for the asymmetry in both stems of the dataset. Additional statistical analyses were carried out using Graphpad Prism 7; details can be found in the respective figure legends.

### Results

IgG Targets

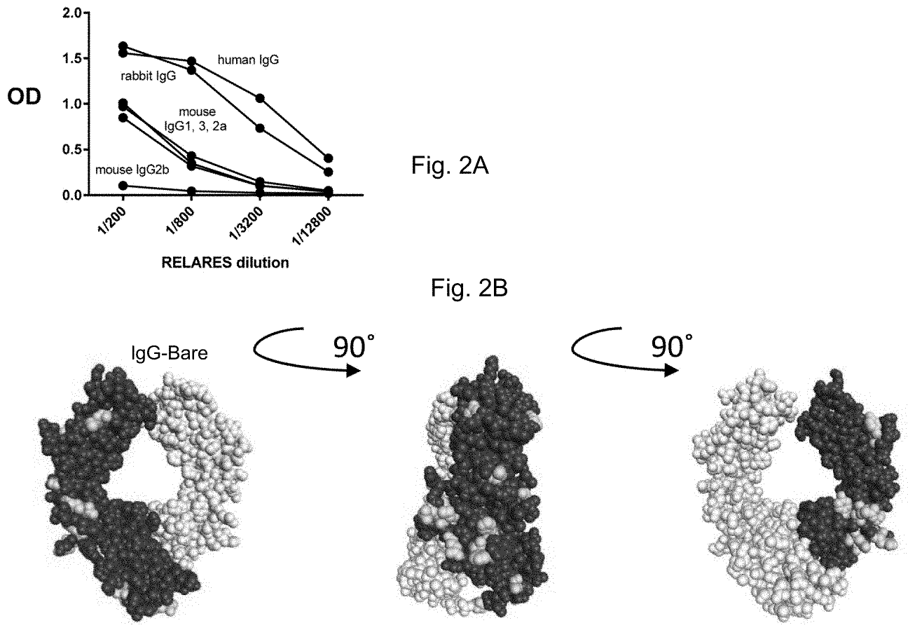

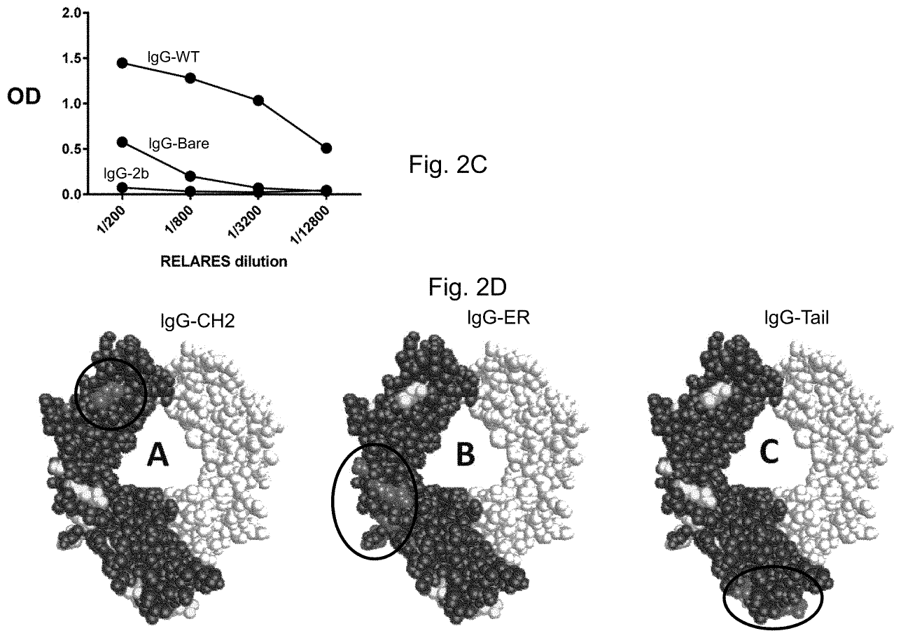

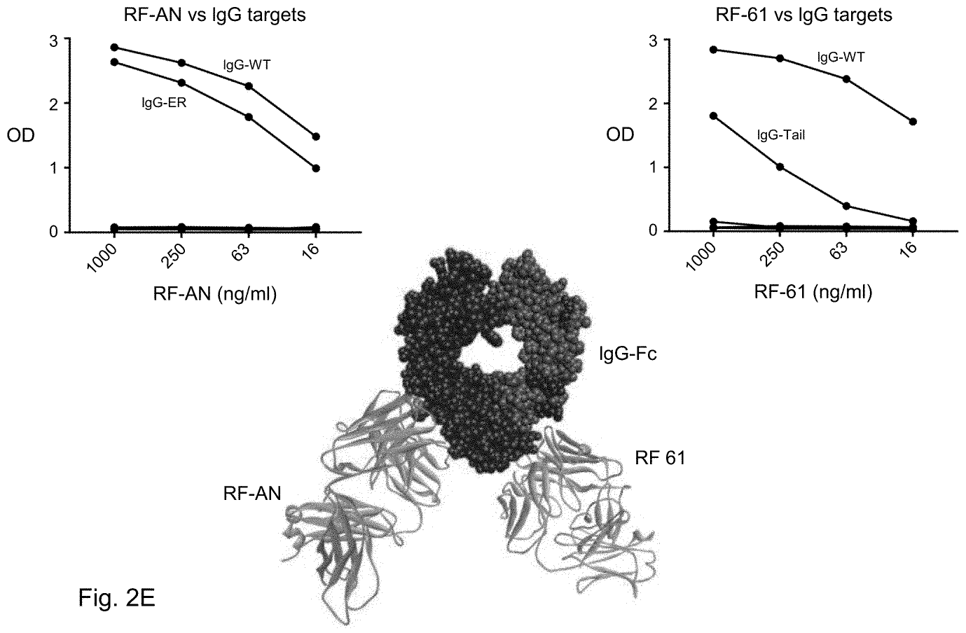

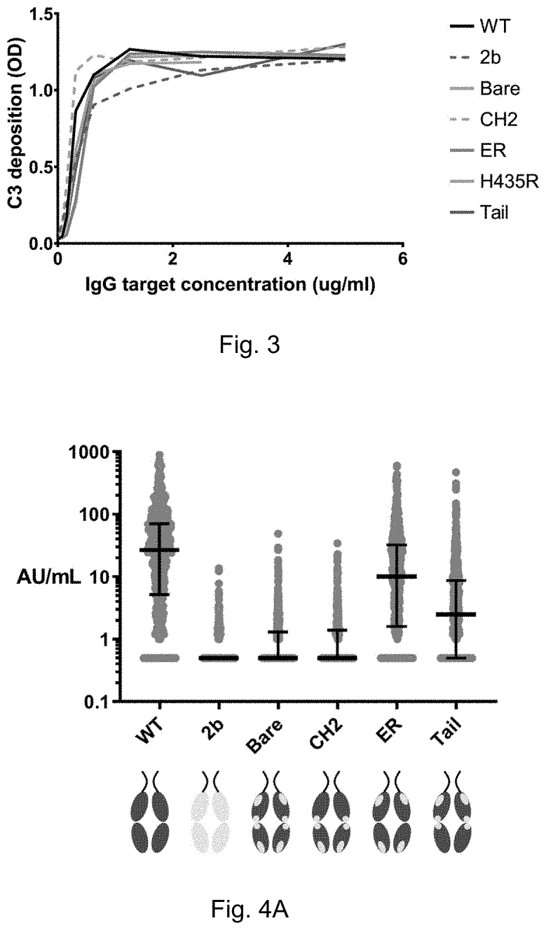

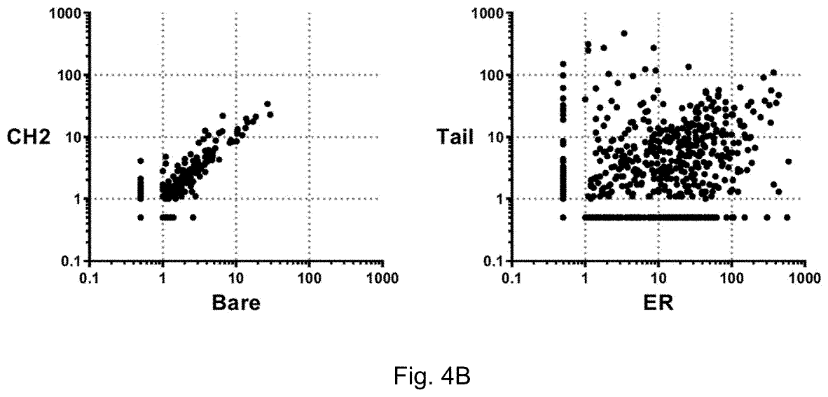

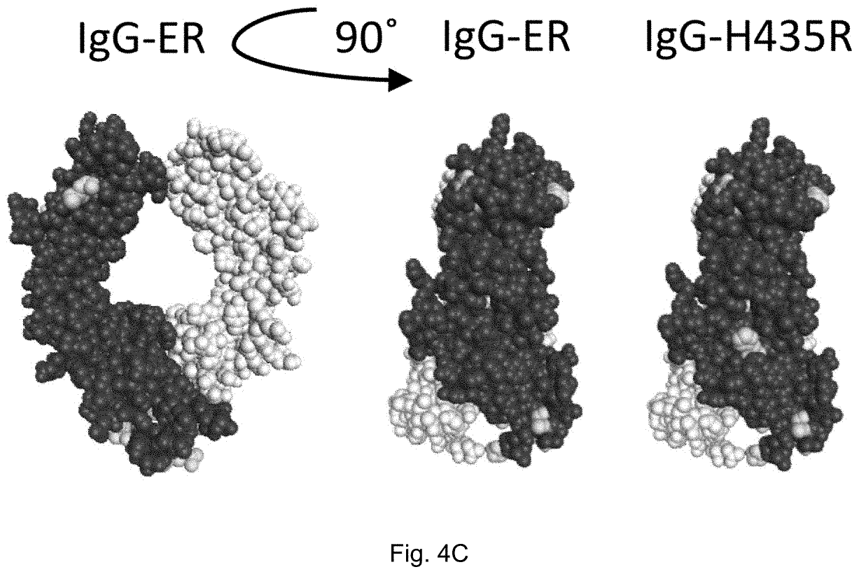

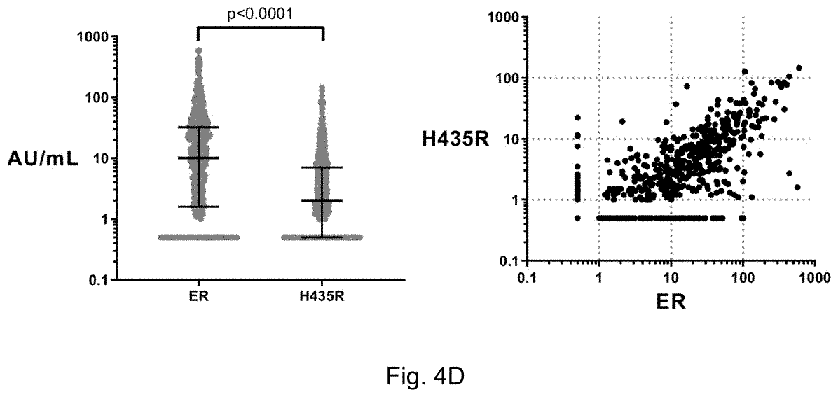

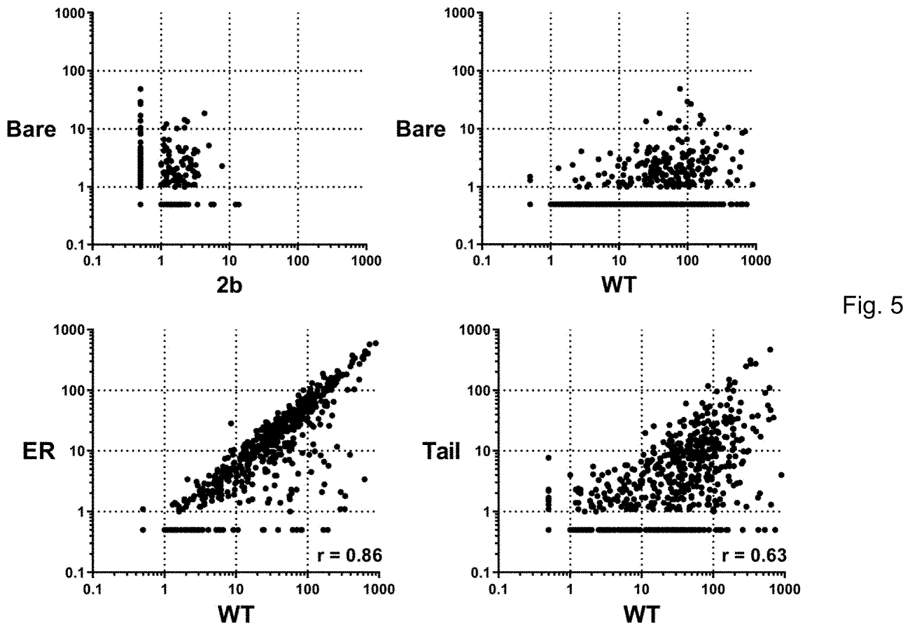

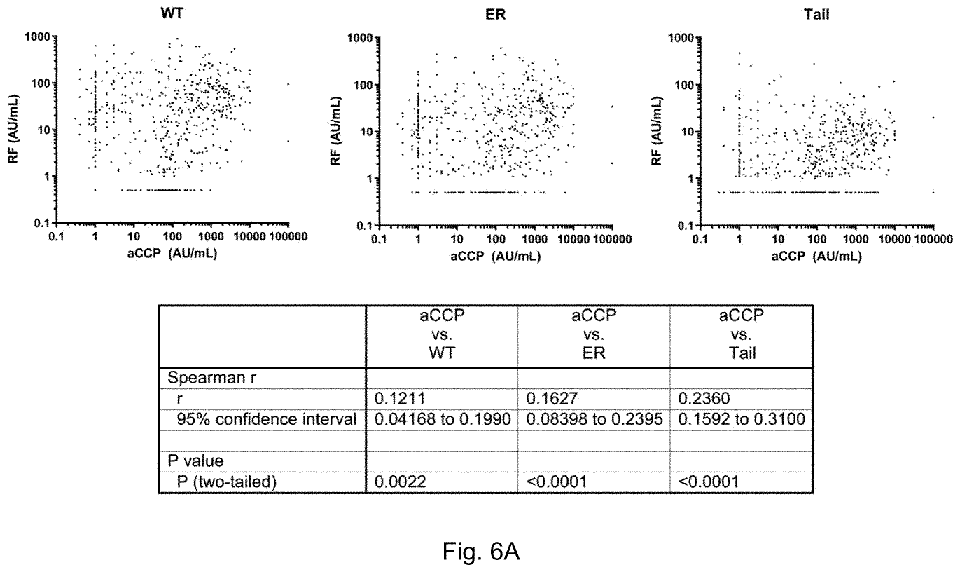

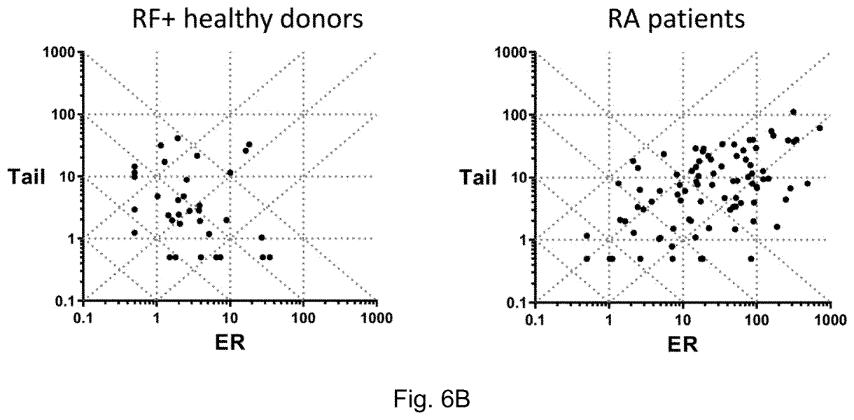

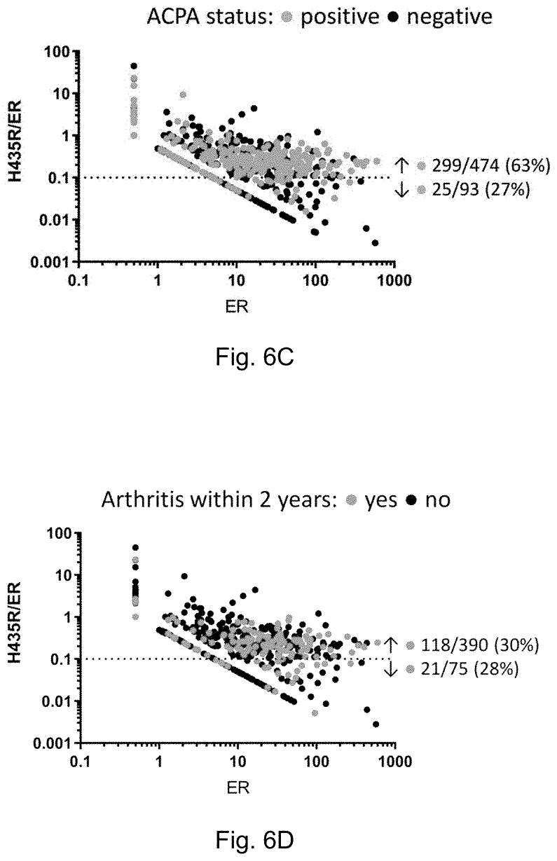

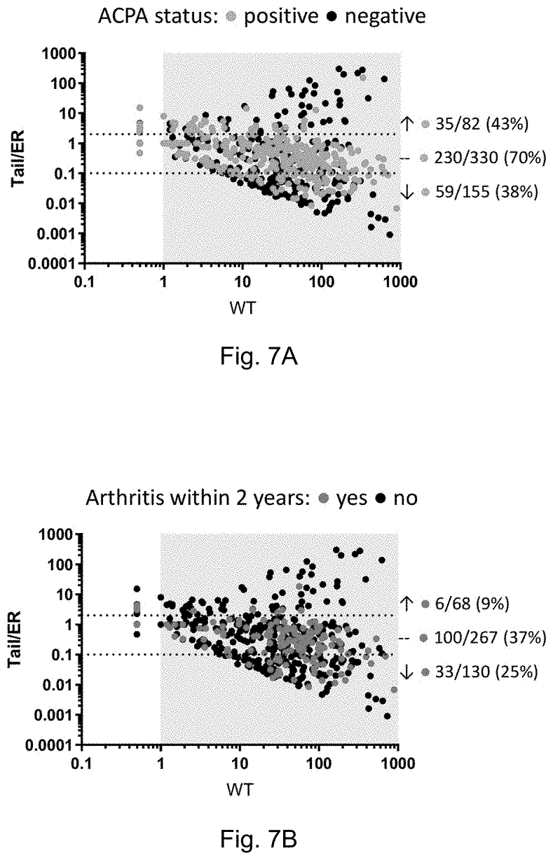

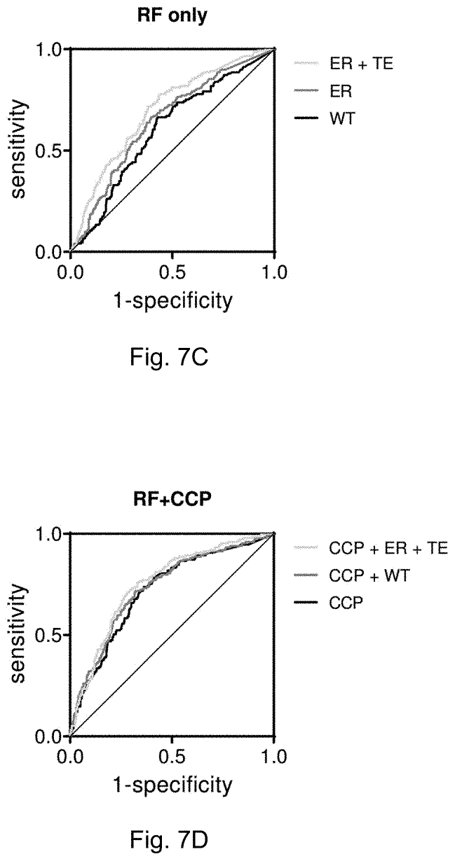

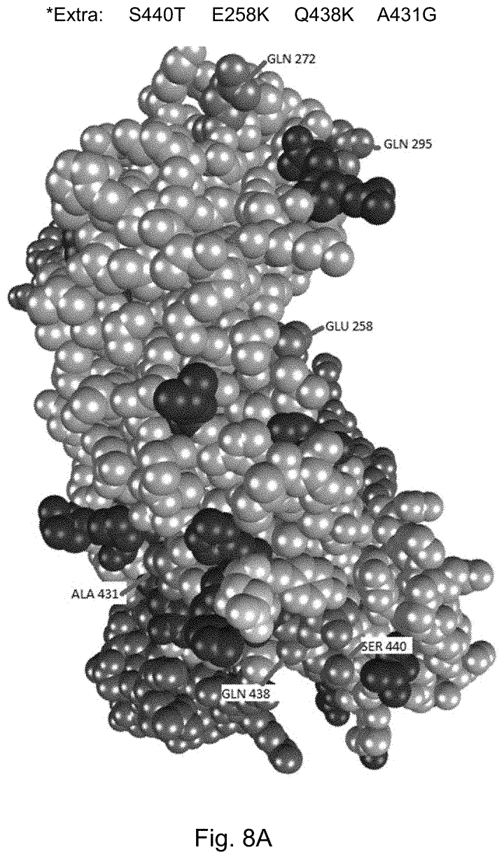

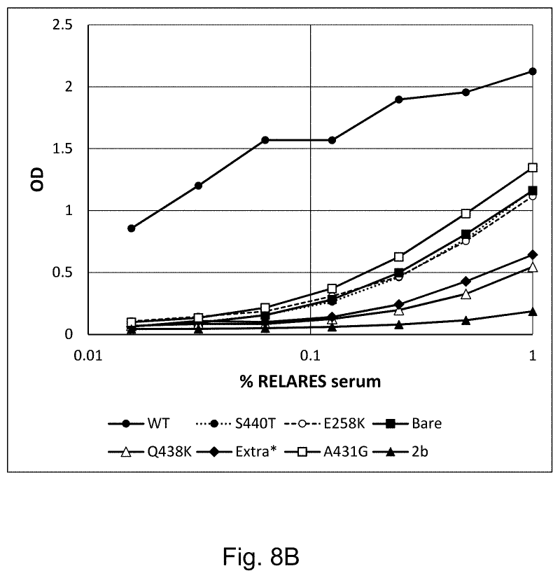

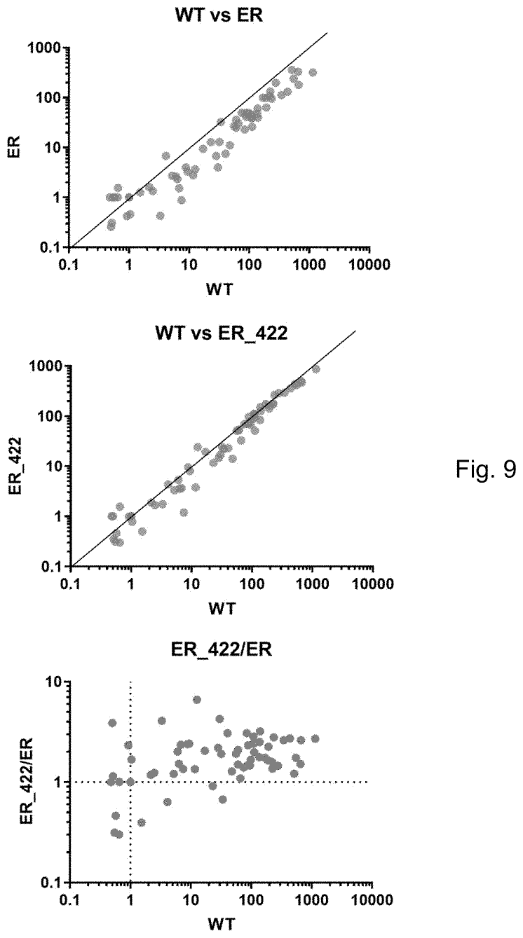

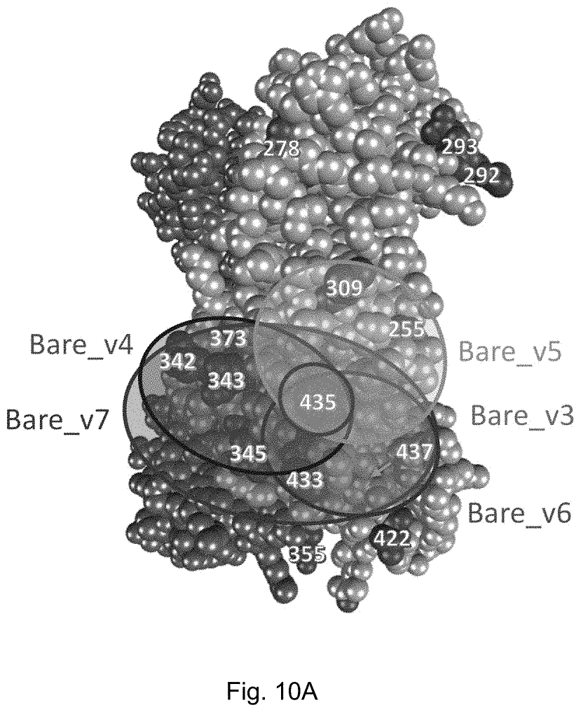

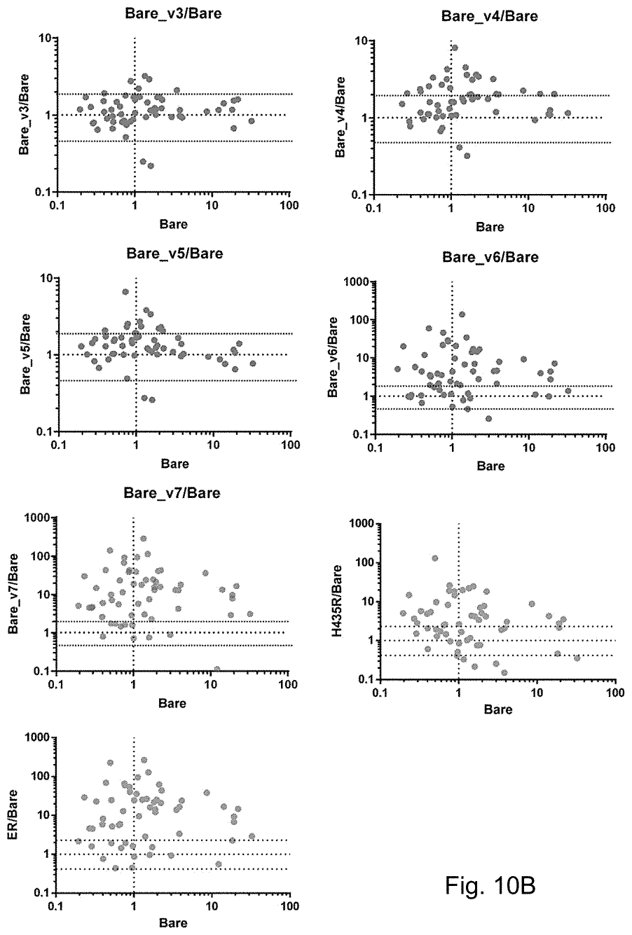

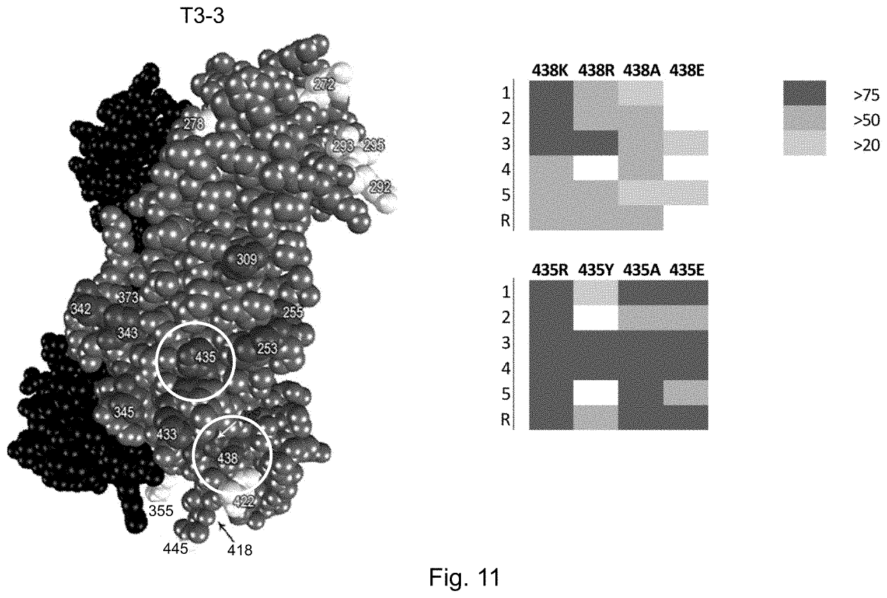

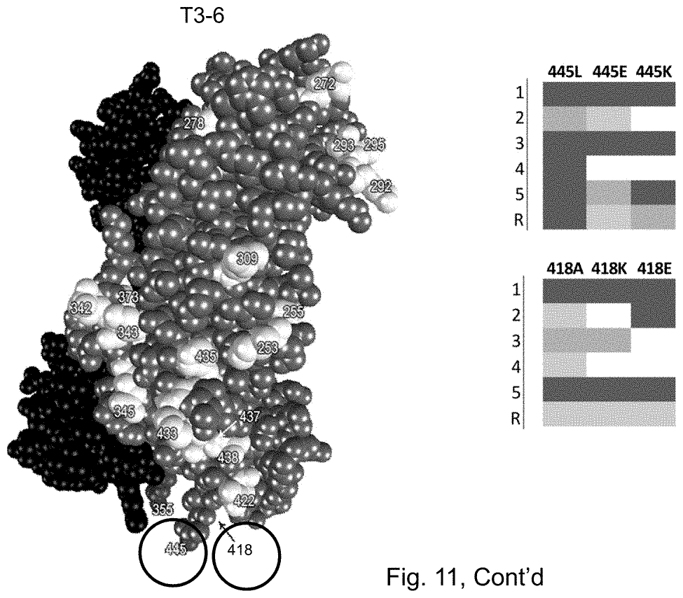

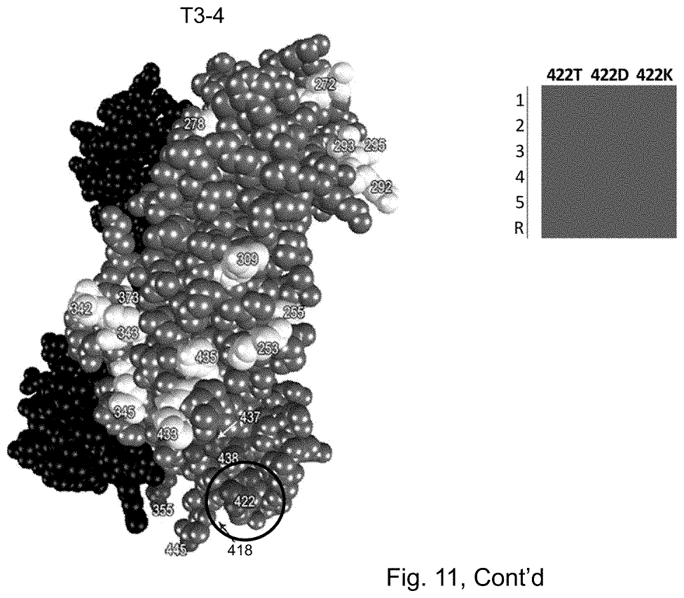

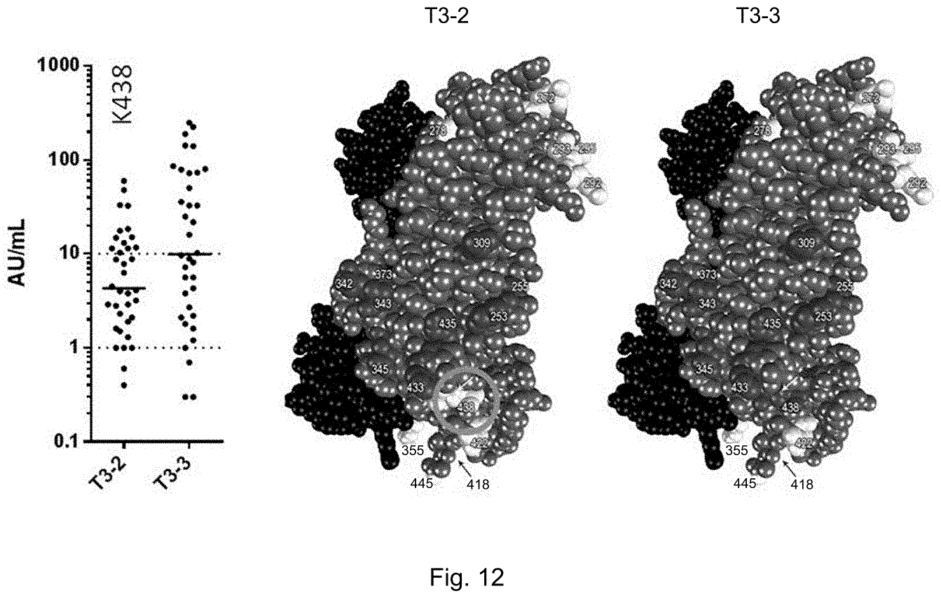

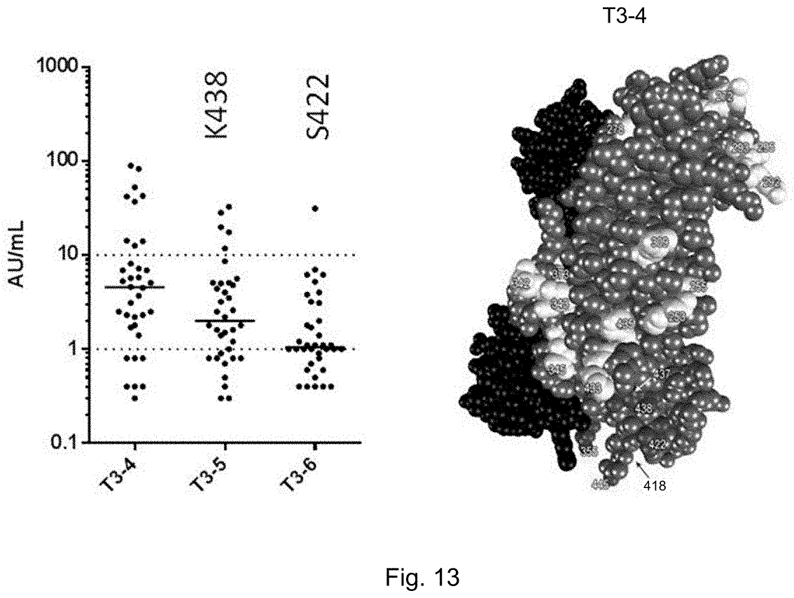

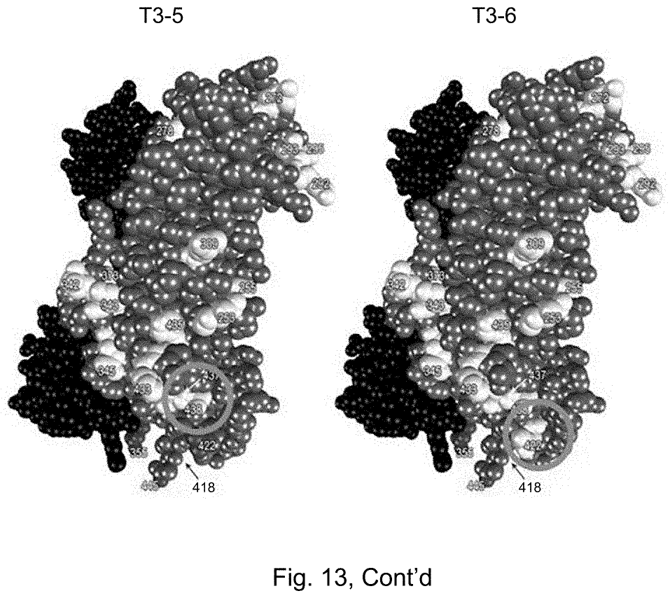

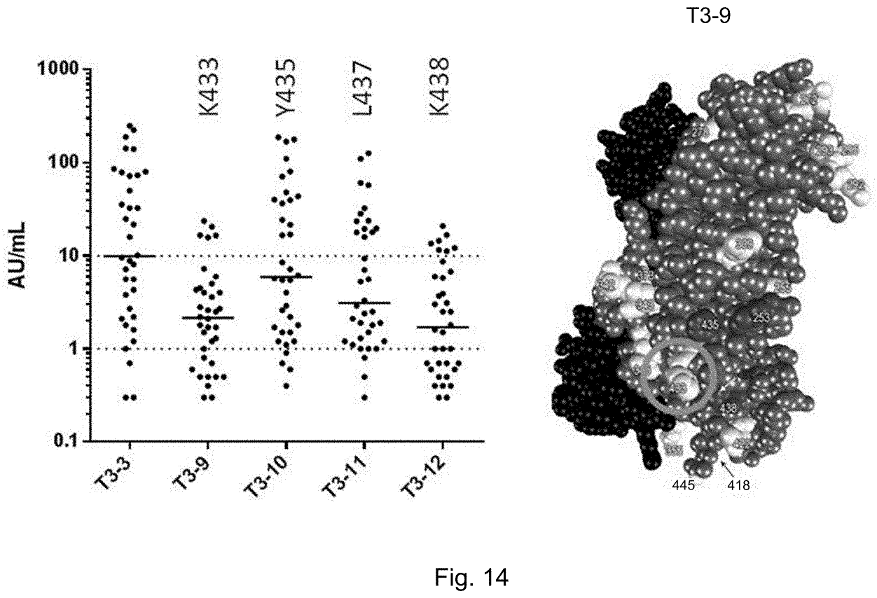

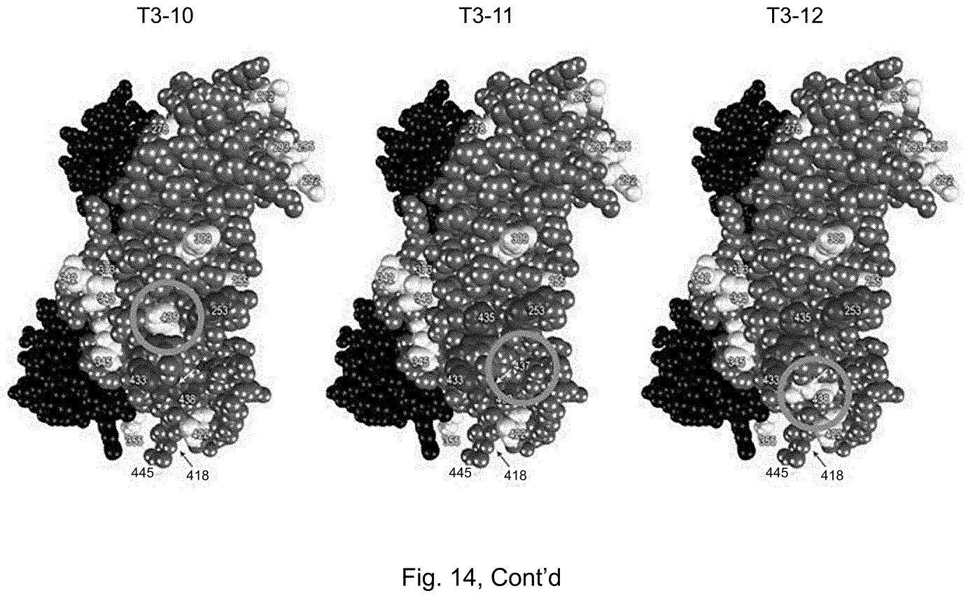

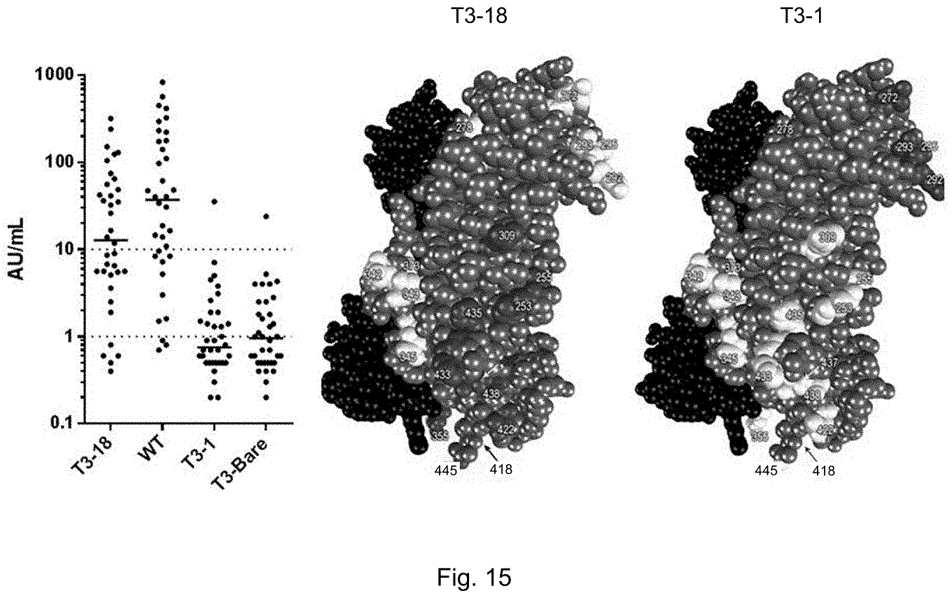

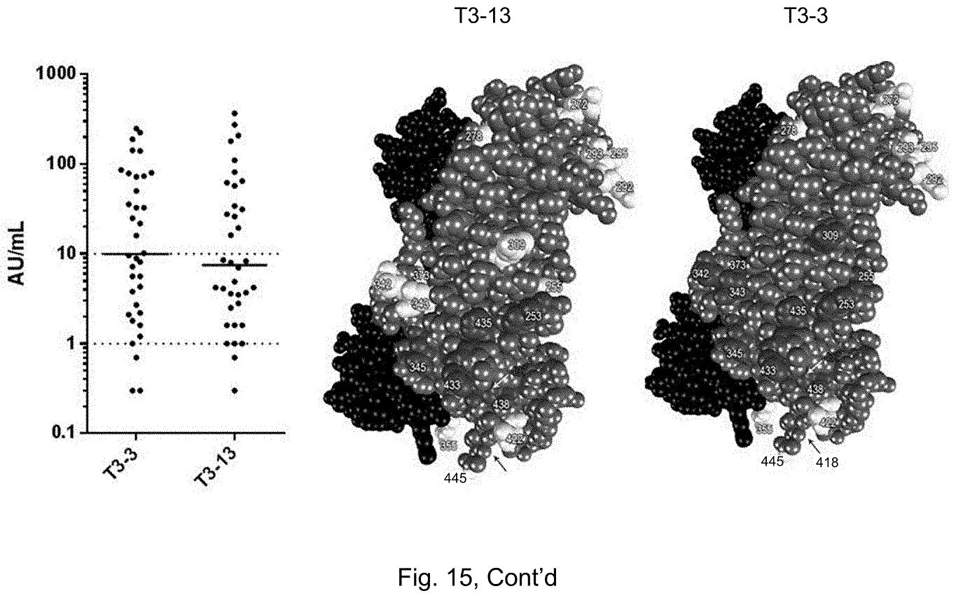

To develop RF assays that can classify RF responses according to their specificities for different IgG-Fc epitopes, several recombinant IgG1 molecules with various amino acid replacements in the Fc domain were engineered. First, a ‘parent’ target was designed to be an IgG1 molecule with most RF reactivity removed. Our starting point was the observation that RFs are cross-reactive to different animal IgGs to vastly different degrees. In particular, cross-reactivity to rabbit IgG is high, whereas cross-reactivity towards mouse IgG is low (Hamako et al. 1995), and particularly low towards mouse IgG2b (FIG. 2A). Comparing the structures of human IgG1, rabbit IgG, and mouse IgG2b yields multiple positions shared by the former two but not the latter. We selected a subset of these, based—amongst others—on solvent exposure, and a human IgG construct was designed and produced with 15 amino acid replacements in the Fc domain where the ‘human amino acid’ was replaced with the ‘mouse amino acid’ as well as an additional P445L mutation (FIG. 2B) (See FIG. 1 for amino acid sequences). In a pooled RF serum standard (see M&M), RF reactivity towards this partially ‘murinized’ IgG, designated IgG-Bare, was indeed found to be much reduced, with circa 2% RF reactivity remaining compared to wild-type IgG1 (IgG1-WT), which corresponds to a conventional RF assay (FIG. 2C).

Next, with IgG-B are as the starting point, three additional recombinant IgG targets were produced where we reintroduced human amino acids in three different clusters, to determine if specific RF reactivity towards these individual clusters could be evaluated: cluster A located in the CH2 domain (‘IgG-CH2’); cluster B at the CH2-CH3 elbow region (‘IgG-ER’); cluster C in the tail region of the CH3 domain (‘IgG-Tail’) (FIG. 2D, FIG. 1). All targets were able to induce complement activation to a similar degree (FIG. 3), indicating that all targets were correctly folded.

Binding properties of the IgG targets were first tested with two monoclonal IgM-RFs: RF61 and RF-AN. The IgG-Fc epitopes bound by RF61 and RF-AN have previously been determined in crystal structure studies (Duquerroy et al. 2007; Corper et al. 1997), which showed that RF-AN binds epitopes in cluster B and RF61 in cluster C. Indeed, while reactivity was lower than against IgG-WT, RF61 bound IgG-Tail but not IgG-ER and vice versa for RF-AN (FIG. 2E), demonstrating differential presence of specific epitopes on the different targets.

Reactivity against engineered IgG targets in seropositive arthralgia patients To test whether the IgG targets could be used to identify distinct RF responses, IgM reactivity against these targets was tested for 639 patients from the Reade seropositive arthralgia cohort. This cohort contains prospectively monitored patients with (a history of) arthralgia who had tested IgM-RF and/or IgG-ACPA positive in conventional assays. In this cohort the development of arthritis is strongly linked to ACPA positivity (Bos et al. 2010). Of the 639 patient samples used in the present study, 214 had tested RF positive, 179 ACPA positive and 187 double-positive; 59 patients had ambiguous antibody status, with levels around the cut-off in the conventional assays or inconsistent results from multiple measurements. These samples were considered ACPA negative for analyses regarding ACPA status (see below).

As shown in FIG. 4A, RF levels in the arthralgia cohort are highest against the wild-type IgG1 target (IgG-WT). Minimal reactivity is seen against the control mouse IgG2b target designated ‘IgG-2b’ (note the logarithmic scale). Compared to IgG-WT, reactivity against IgG-Bare is also low, indicating that the most important hotspots for RF binding on IgG-Fc were successfully disrupted with the 16 amino acid mutations and that the number of RF epitopes on IgG-Fc is limited. Nevertheless, there is more residual reactivity against IgG-Bare than against IgG-2b on a group level and a small number of patients show substantially more anti-Bare than anti-2b reactivity (FIG. 5), primarily in samples with high RF (anti-IgG-WT) levels.

Reactivity against cluster A (IgG-CH2)—which overlaps with the region where most Fc receptors bind to IgG—appears to be low in most patients, and since there is a tight correlation between reactivity against IgG-CH2 and IgG-B are it may be concluded that the Fc receptor bind region is hardly specifically targeted by RF responses (FIG. 4B). Reactivity towards cluster B (IgG-ER) comprises the largest part of the overall RF binding. Reactivity towards cluster C (IgG-Tail) is variable and correlation with the overall reactivity towards WT is much weaker than for cluster B and WT (FIG. 5). Since cluster B and C are spatially removed from each other we hypothesized that we would be able to find separate RF responses against these two clusters. Indeed, when we compare anti-IgG-ER and anti-IgG-Tail reactivity we find samples with RF reactivity skewed towards either one target, but also many samples with RF reactivity against both (FIG. 4B).

Cluster B is situated at the CH2-CH3 elbow region and putatively corresponds to those parts of the Fc region that are important for Ga reactivity (or reactivities). To be able to determine how much of the reactivity against cluster B corresponds to ‘classic’ (i.e., H435-dependent) Ga reactivity, an additional IgG target was produced that differs from IgG-ER only at position 435, with an arginine (R) replacing the histidine (H): IgG-H435R (FIG. 4C). FIG. 4D shows that for almost all arthralgia patients with Ga reactivity (i.e. anti-IgG-ER levels ≥1), Ga reactivity is at least partly dependent on the presence of H435 in the Fc domain, with a median loss of reactivity of 76% when H435 is mutated compared to IgG-ER. These data suggest that classic Ga reactivity is an important part of the RF response in almost all patients.

Taken together, these results demonstrate the feasibility of dissecting reactivity patterns of RF responses using our new methodology.

Association of RF reactivity pattern with ACPA status and clinical outcome Next, we investigated whether in the arthralgia patients RF reactivity patterns could be identified that are associated with ACPA status and clinical outcome. First we analyzed whether levels of reactivity against the individual targets correlated with ACPA (i.e. anti-CCP2) levels. A rather weak correlation was found for RF reactivity against IgG-WT versus anti-CCP (Spearman's r=0.12, p=0.002), with a stronger correlation for the IgG-ER and IgG-Tail targets (ER vs a-CCP r=0.16 p <0.0001, Tail vs a-CCP r=0.24 p <0.0001) (FIG. 6A).

As mentioned, earlier studies suggested that a broader RF response indicates a more pathogenic, RA-associated RF response. In line with these studies, comparison of two smaller datasets, one consisting of RF+ healthy donors, the other of established RA patients, indicated that RF responses in RA patients show a broader anti-IgG reactivity than HDs, with substantial reactivity against both IgG-ER and IgG-Tail in many RA patients compared to reactivity against mainly one of these targets in many HDs (FIG. 6B). Therefore, we hypothesized that in our seropositive arthralgia cohort a similarly skewed, ‘HD-like’ RF reactivity pattern associates with low ACPA positivity and little arthritis development.

Association with ACPA status was tested by plotting the Tail/ER ratio, to visualize skewedness of RF reactivity towards either target, versus WT and comparing ACPA positive and negative patients. FIG. 7A shows that strong skewing of the RF response towards either Tail or ER is indeed associated with ACPA negativity. For example, the subsets of patients with either a high (>2) or low (≤0.1) Tail/ER ratio predominantly belong to the ACPA negative group, whereas the majority (70%) of patients that have reactivity against both targets are ACPA positive. Furthermore, we find significantly less ACPA positivity in patients with a RF reactivity pattern dominated by classic Ga reactivity (defined as H435R/ER ratio ≤0.1) (25/93=27% ACPA positivity versus 299/474=63%) (Supp. FIG. 4C). This is in accordance with earlier studies suggesting that RFs from RF-positive healthy donors mainly show classic Ga reactivity.

Next, we determined whether RF reactivity pattern is associated with development of arthritis. We analyzed 465 arthralgia patients with an RF level against IgG-WT ≥1 and a follow-up of two years. Of these, 139 patients (30%) developed arthritis within two years, with a median time to arthritis of 6.9 months. As may be judged from FIG. 7B, an RF reactivity pattern skewed towards anti-Tail or anti-ER is associated with a lower risk of progression to arthritis. Using the same example as above (i.e., arbitrarily defining skewedness as Tail/ER ≤0.1 or Tail/ER >2), 198/465 patients (43%) show a skewed RF response. Of these 198 patients, 39 (=20%) developed arthritis within 2 years, versus 100/267 (=37%) in the patients with a Tail/ER ratio between 0.1 and 2.

Logistic regression was carried out to evaluate the association between autoantibody status and development of arthritis within 2 years (Ar2yrs) in more detail. RF reactivity against IgG-WT on its own is significantly associated with Ar2yrs (p<0.0001) and ROC analysis yields an AUC of 0.603 (FIG. 7C). Individually, ER and Tail are also significantly associated with Ar2yrs (p<0.0001). Using ER, an AUC of 0.646 is obtained, suggesting an improved association with Ar2yrs over WT; this association becomes stronger for the combination of ER and Tail. Combining ER titer and a normalized Tail/ER ratio (TE) as a measure of skewness towards one of these targets results in an AUC of 0.697; both parameters are significantly correlated (p<0.001). Anti-CCP on its own yields an AUC of 0.718 (p<0.0001) (FIG. 7D). Combination of a-CCP with WT results in enhanced association with Ar2yrs (AUC 0.732; p <0.001); combination of a-CCP with ER and TE results in a further enhanced association with Ar2yrs (AUC 0.753; p <0.001 & p=0.016 for TE); the latter model has significantly improved predictive power over the former as evaluated using the Vuong test (p=0.007).

Although ‘classic Ga reactivity’ (H435R/ER ratio ≤0.1) was associated with low ACPA positivity (see above), this reactivity patterns did not associate with a lower rate of arthritis development within 2 years (FIG. 6D).

Overall, these data suggest that a broader RF response, characterized by substantial reactivity against more epitopes, is associated with the presence of ACPAs and a higher probability of developing arthritis.

Additional Targets: Positions 422 and 438

As shown in FIG. 8, mutation Q438K results in further reduction of reactivity towards the bare target. Compared to bare, the Q438K target shows a further diminished reactivity of the Relares multi-patient RF serum pool; demonstrating that position 438 is an important determinant for a subset of RF reactivity in RA patients.

FIG. 9 demonstrates that position 422 is an important determinant for a subset of RF reactivity in RA patients. Comparison of top left and right panels shows that the ER_422 target (which is the ‘ER’ target, but with V422 instead of 5422) captures much more reactivity than the ER target in comparison to WT (note the logarithmic scale); demonstrating that position 422 is an important determinant for a subset of RF reactivity in RA patients.

Further Dissecting the ER Cluster

Reactivities to targets based on the ‘bare’ target, with subsets of the ER cluster of mutations reverted to human were determined. The different clusters (Bare_v3-Bare_v7) are schematically indicated in the structure model in FIG. 10. All of these positions together make up the ‘ER’ cluster. The data panels in FIG. 10 show reactivity to the respective targets, expressed as ratio (Bare_vx/Bare) and plotted against residual reactivity to the Bare target in sera of patients with established RA. Note that whereas reactivity to the ER cluster is reduced for the ‘ER’ target with an H435R mutation, the ‘inverse’ Bare_v3 structure (H435 but all other positions in the ‘ER’ cluster mutated) shows little reactivity. This demonstrates that position is an important determinant of many epitopes in the ER region, but not in itself sufficient.

Furthermore, reactivity to three other clusters (4, 5, and 6) show various amounts of reactivity in these sera, with most reactivity to the Bare_v6, comprising positions 433, 435, and 437). Therefore, this triad of AA positions defines an important region of RF reactivity.

Replacement of Amino Acids Residues by Varying Amino Acids on Binding of RF

To explore the impact of mutations towards mouse amino acids and other than the IgG2b mouse equivalent, mutations covering amino acids of different categories (non-polar, polar, positively charged and/or negatively charged) were introduced in several targets at the indicated locations (positions 438, 435, 422, 418, and 445), as detailed in table 1.

TABLE 1
Tested amino acid mutations.

| | | | other tested amino |
|---|---|---|---|
| position | human | mouse | acids |
| 418 | glutamine (Q) | glutamic acid (E) | alanine (A) |
| | | | lysine (K) |
| 422 | valine (V) | serine (S) | threonine (T) aspartic |
| | | | acid (D) lysine (K) |
| 435 | histidine (H) | tyrosine (Y) | arginine (R) |
| | | | alanine (A) |
| | | | glutamic acid (E) |
| 438 | glutamine (Q) | lysine (K) | arginine (R) |
| | | | alanine (A) |
| | | | glutamic acid (E) |
| 445 | proline (P) | leucine (L) | glutamic acid (E) |
| | | | lysine (K) |

In all cases, all mutations reduced RF binding in at least a subset of sera from RA patients (1-5) in comparison to the non-mutated, human equivalent (FIG. 11). R indicates a pool of RF sera.

Impact of Mutation of Individual Positions or Combinations of 2 Positions on RF Binding

Different targets were prepared in which individual amino acids or combinations of amino acids in one epitope were mutated to assess the impact of these mutations on RF binding.

Mutation of position 438 as well as 422 reduce RF reactivity in the context of Tail epitope (FIGS. 12 and 13).

A series of matching targets wherein positions 433, 435, 437, and 438 were mutated was analysed for binding of RFs from RA patients to demonstrate the impact of individual mutations (FIG. 14). The binding to these targets differs depending on which of the amino acids is mutated, but all cause a significant reduction of RF binding. For sake of comparison, reactivity to T3-3 is also shown.

The cluster of 342/343/345/373 impacts the binding of RF—as inferred from comparison of T3-18 vs WT (FIG. 15). The contribution of the CH2 epitope (T3-1 vs T3-Bare) to RF binding is very small (although for a small subset of individual sera it can make a difference), which justifies the comparison of T3-18 to WT. A particular contribution is provided by position 345, as can be inferred from comparison of T3-13 to T3-3, in the context of the above.

### REFERENCES

Allen J C, Kunkel H G. Hidden rheumatoid factors with specificity for native gamma globulins. Arthritis Rheum. 1966; 9(6):758-68.
Bagci H, Kohen F, Kuscuoglu U, Bayer E A, Wilchek M. Monoclonal anti-biotin antibodies simulate avidin in the recognition of biotin. FEBS Lett. 1993; 322(1):47-50.
Berman H M, Westbrook J, Feng Z, Gilliland G, Bhat T N, Weissig H, et al. The Protein Data Bank. Nucleic acids research. 2000; 28(1):235-42.
Bonagura V R, Artandi S E, Davidson A, Randen I, Agostino N, Thompson K, et al. Mapping studies reveal unique epitopes on IgG recognized by rheumatoid arthritis-derived monoclonal rheumatoid factors. Journal of immunology. 1993; 151(7):3840-52.
Bonagura V R, Agostino N, Borretzen M, Thompson K M, Natvig J B, Morrison S L. Mapping IgG epitopes bound by rheumatoid factors from immunized controls identifies disease-specific rheumatoid factors produced by patients with rheumatoid arthritis. J Immunol. 1998; 160(5):2496-505.
Bos W H, Wolbink G J, Boers M, Tijhuis G J, de Vries N, van der Horst-Bruinsma I E, et al. Arthritis development in patients with arthralgia is strongly associated with anti-citrullinated protein antibody status: a prospective cohort study. Annals of the rheumatic diseases. 2010; 69(3):490-4.
Corper A L, Sohi M K, Bonagura V R, Steinitz M, Jefferis R, Feinstein A, et al. Structure of human IgM rheumatoid factor Fab bound to its autoantigen IgG Fc reveals a novel topology of antibody-antigen interaction. Nat Struct Biol. 1997; 4(5):374-81.
Duquerroy S, Stura E A, Bressanelli S, Fabiane S M, Vaney M C, Beale D, et al. Crystal structure of a human autoimmune complex between IgM rheumatoid factor RF61 and IgG1 Fc reveals a novel epitope and evidence for affinity maturation. JMolBiol. 2007; 368(5):1321-31.
Edelman G M et al. The covalent structure of an entire gammaG immunoglobulin molecule. Proc Natl Acad Sci USA. 1969; 63(1):78-85
Falkenburg W J J, Kempers A C, Dekkers G, Ooijevaar-de Heer P, Bentlage A E H, Vidarsson G, et al. Rheumatoid factors do not preferentially bind to ACPA-IgG or IgG with altered galactosylation. Rheumatology. 2017; 56(11):2025-30.
Falkenburg W J, van Schaardenburg D, Ooijevaar-de Heer P, Wolbink G, Rispens T. IgG Subclass Specificity Discriminates Restricted IgM Rheumatoid Factor Responses From More Mature Anti-Citrullinated Protein Antibody-Associated or Isotype-Switched IgA Responses. Arthritis & rheumatology. 2015; 67(12):3124-34.
Falkenburg W J J, von Richthofen H J, Rispens T. On the origin of rheumatoid factors: Insights from analyses of variable region sequences. Semin Arthritis Rheum. 2018 Jun. 24. pii: S0049-0172(18)30241-5. doi: 10.1016/j.semarthrit.2018.06.006. [Epub ahead of print]
Hamako J, Ozeki Y, Matsui T, Yamamoto Y, Inoue T, Yukitake J, et al. Binding of human IgM from a rheumatoid factor to IgG of 12 animal species. Comparative biochemistry and physiology Part B, Biochemistry & molecular biology. 1995; 112(4):683-8.
Kohen F, Bagci H, Barnard G, Bayer E A, Gayer B, Schindler D G, et al. Preparation and properties of anti-biotin antibodies. Methods Enzymol. 1997; 279:451-63.
Klein F, Janssens M B. Standardisation of serological tests for rheumatoid factor measurement. AnnRheumDis 1987; 46(9):674-80.
Peterson C, Malone C C, Williams R C, Jr. Rheumatoid-factor-reactive sites on CH3 established by overlapping 7-mer peptide epitope analysis. Molecular immunology. 1995; 32(1):57-75.
Sivalingam G N, Shepherd A J. An analysis of B-cell epitope discontinuity. Molecular immunology. 2012; 51(3-4):304-9.
Williams R C, Jr., Malone C C. Rheumatoid-factor-reactive sites on CH2 established by analysis of overlapping peptides of primary sequence. Scandinavian journal of immunology. 1994; 40(4):443-56.

1. A method for typing rheumatoid factor (RF), comprising:
contacting a sample from a subject, said sample comprising or suspected of comprising RF, with at least one compound comprising a recombinant human IgG class fragment crystallizable (Fc) domain, wherein said Fc domain comprises:
1) a first RF epitope wherein amino acid H435 and one or more amino acids selected from the group consisting of R255, L309, Q342, P343, E345, Y373, H433, T437, and Q438 of said IgG Fc domain are replaced by another amino acid, and
2) a second RF epitope comprising amino acid V422 and optionally one or more amino acids selected from the group consisting of R355, Q418, Q438, and P445 of said IgG Fc domain, or wherein said compound comprises;
1) a first RF epitope wherein amino acid V422 and optionally one or more amino acids selected from the group consisting of R355, Q418, Q438, and P445 of said IgG Fc domain are replaced by another amino acid, and
2) a second RF epitope comprising amino acid H435 and one or more amino acids selected from the group consisting of R255, L309, Q342, P343, E345, Y373, H433, T437, and Q438 of said IgG Fc domain, or
wherein in said compound amino acids H435 and V422 and one or more amino acids selected from the group consisting of R255, Q342, P343, E345, Y373, H433 and T437 are replaced by another amino acid and the compound comprises one or more amino acids selected from the group consisting of R255, L309, Q342, P343, E345, Y373, H433, T437, R355, Q418 and P445, and
determining binding of RF to said at least one compound, wherein the amino acid numbering is according to EU numbering.

2. The method according to claim 1, wherein:
said first RF epitope comprises amino acids at positions 433, 435, 437 255, 309, 342, 343, 345 and 373 of said IgG Fc domain and said second RF epitope comprises amino acids at position 355, 418, 422 and 445 of said IgG Fc domain, or said first RF epitope comprises amino acids a positions 355, 418, 422 and 445 of said IgG Fc domain and said second RF epitope comprises amino acids at positions 433, 435, 437, 255, 309, 342, 343, 345 and 373 of said IgG Fc domain wherein said amino acid numbering is according to EU numbering.

3. The method according to claim 1 or 2, wherein said compound comprises:
a first RF epitope wherein amino acids H433, H435 and T437 and optionally one or more amino acids selected from the group consisting of R255, L309,
Q342, P343, E345 and Y373 of said IgG Fc domain are replaced by another amino acid, or
a first RF epitope wherein amino acids R355, Q418 and V422 of said IgG Fc domain are replaced by another amino acid.

4. The method according to claim 1 or 2, wherein said compound comprises amino acid H435 and optionally amino acids H433 and/or T437, and wherein amino acids V422, one or more amino acids selected from the group consisting of R255, L309, Q342, P343, E345 and Y373 and optionally one or more amino acids selected from the group consisting of R355, Q418, Q438 and P445 are replaced by another amino acid.

5. The method according to claim 4, wherein said compound comprises amino acids H433, H435 and T437 and wherein amino acids R255, L309, Q342, P343, E345, Y373, R355 Q418, V422 and P445, and optionally Q438 are replaced by another amino acid.

6. The method according to claim 1 or 2, wherein said compound comprises amino acid H435 and optionally one or more amino acids selected from the group consisting of R255, L309, Q342, P343, E345 and Y373 and
wherein amino acids V422, one or both amino acids selected from H433 and T437 and optionally one or more amino acids selected from the group consisting of R355, Q418, Q438, and 445 are replaced by another amino acid. The method according to claim 6, wherein said compound comprises amino acids R255, L309, Q342, P343, E345, Y373 and H435 and wherein in said compound amino acids R355, Q418, V422, H433, T437 and P445 are replaced by another amino acid.

7. The method according to claim 1 or 2, wherein amino acids H435 and V422, and optionally Q438 have been replaced by another amino acid, and wherein:
R255 and L309 are replaced by another amino acid and the compound comprises amino acids Q342, P343, E345, Y373, H433, T437, R355, Q418 and P445, or
Q342, P343, E345 and Y373 are replaced by another amino acid and the compound comprises amino acids R255, L309, H433, T437, R355, Q418 and P445, or H433 and T437 are replaced by another amino acid and the compound comprises amino acids R255, L309, Q342, P343, E345, Y373, R355, Q418 and P445, or
R355, Q418 and P445 are replaced by another amino acid and the compound comprises amino acids R255, L309, Q342, P343, E345, Y373, H433 and T437.

9. The method according to claim 1 or 2, wherein in said compound:
amino acids H433, H435 and T437 are replaced by another amino acid and optionally one or more amino acids selected from the group consisting of 8255, L309, Q342, P343, E345 and Y373 are replaced by another amino acid (compound A1), or
amino acids H433, H435 and T437 are replaced by another amino acid and the compound comprises amino acids R255, L309, Q342, P343, E345 and Y373 (compound A2), or
amino acids R255, L309 and H435, are replaced by another amino acid and the compound comprises amino acids Q342, P343, E345, Y373, H433, and T437 (compound A3), or
amino acids Q342, P343, E345, Y373 and H435 are replaced by another amino acid and the compound comprises amino acids R255, L309, H433 and T437 (compound A4), or
amino acids R255, L309, H433 H435 and T437 are replaced by another amino acid and the compound comprises amino acids Q342, P343, E345 and Y373 (compound A5), or amino acids R255, L309, Q342, P343, E345, Y373, H433, H435 and T437 are replaced by another amino acid (compound A6), or
amino acid V422 is replaced by another amino acid and the compound comprises amino acids R355, Q418 and P445 (compound B2), or
amino acids R355, Q418 and V422 are replaced by another amino acid and the compound comprises amino acid P445 (compound B3), or
amino acids R355, Q418, V422 and P445 are replaced by another amino acid (compound B4), or
amino acids V422, one or more amino acids selected from the group consisting of R255, L309, Q342, P343, E345 and Y373 and optionally one or more amino acids selected from the group consisting of R355, Q418, Q438 and P445 are replaced by another amino acid and the compound comprises amino acid H435 and optionally H433 and/or T437 (compound C), or
amino acids R255, L309, Q342, P343, E345, Y373, R355 Q418, V422 and 445 are replaced by another amino acid and the compound comprises amino acids H433, H435 and T437 (compound C1), or
amino acid V422, one or both amino acids selected from H433 and T437 and optionally one or more amino acids selected from the group consisting of R355, Q418, Q438 and P445 are replaced by another amino acid and the compound comprises amino acid H435 and optionally one or more amino acids selected from the group consisting of R255, L309, Q342, P343, E345 and Y373 (compound D), or
amino acids R355, Q418, V422, H433, T437 and P445 are replaced by another amino acid and the compound comprises amino acids R255, L309, Q342, P343, E345, Y373 and H435 (compound D1), or
at least two amino acids selected from the group consisting of R255, L309, Q342, P343, E345, Y373, V422, H433, H435 and T437 are replaced by another amino acid and the compounds comprises one or more amino acids selected from the group consisting of R255, L309, Q342, P343, E345, Y373, H433, H435, T437, R355, Q418, V422 and P445 (compound E), or amino acids H435 and/or V422 and one or more amino acids selected from the group consisting of R255, L309, Q342, P343, E345, Y373, H433 and T437 are replaced by another amino acid and the compound comprises one or more amino acids selected from the group consisting of R255, L309, Q342, P343, E345, Y373, H433, H435, T437, R355, Q418, V422 and P445 (compound E1), or amino acids H435 and V422 and optionally one or more amino acids selected from the group consisting of R255, L309, Q342, P343, E345, Y373, H433 and T437 are replaced by another amino acid and the compound comprises one or more amino acids selected from the group consisting of R255, L309, Q342, P343, E345, Y373, H433, T437, R355, Q418 and P445 (compound E2), or amino acids H435 and V422, and optionally amino acid Q438, are replaced by another amino acid and the compound comprises amino acids R255, L309, Q342, P343, E345, Y373, H433, T437, R355, Q418 and P445 (compound E3), or amino acids H435, V422, R255 and L309 are replaced by another amino acid and the compound comprises amino acids Q342, P343, E345, Y373, H433, T437, R355, Q418 and P445 (compound E4), or amino acids H435, V422, Q342, P343, E345 and Y373 are replaced by another amino acid and the compound comprises amino acids R255, L309, H433, T437, R355, Q418 and P445 (compound E5), or amino acids H435, V422, H433 and T437 are replaced by another amino acid and the compound comprises amino acids R255, L309, Q342, P343, E345, Y373, R355, Q418 and P445 (compound E6), or amino acids H435, V422, R355, Q418 and P445 are replaced by another amino acid and the compound comprises amino acids R255, L309, Q342, P343, E345, Y373, H433 and T437 (compound E7), or two or more amino acids selected from the group consisting of H435, Q418, R355, P445, R255 and L309 are replaced by another amino acid and the compound comprises one or more amino acids selected from the group consisting of H433, T437, Q438 and V422, or
amino acids H435, Q418, R355 and P445 are replaced by another amino acid and the compound comprises amino acids H433, T437, Q438 and V422, or
one or more amino acids selected from the group consisting of H433, T437, Q438 and V422 are replaced by another amino acid and the compound comprises two or more amino acids selected from the group consisting of H435, R255 and L309, or
amino acids H433, T437, Q438 and V422 are replaced by another amino acid and the compound comprises amino acids H435, Q418, R355 and P445, or
amino acids Q418, R355 and P445 are replaced by another amino acid and the compound comprises amino acid V422 and two or more amino acids selected from the group consisting of R255, L309, Q342, P343, E345, Y373, H433, H435 and T437, or
amino acid V422 and two or more amino acids selected from the group consisting of R255, L309, Q342, P343, E345, Y373, H433, H435 and T437 are replaced by another amino acid and the compound comprises amino acids Q418, R355 and P445.

10. The method according to any one of claims 1-9, further comprising contacting said sample with at least one compound comprising a recombinant human IgG class fragment crystallizable (Fc) domain, wherein:
amino acids R255, Y278, R292, E293, L309, Q342, P343, E345, R355, Y373, Q418, V422, H433, H435, T437 and P445 are replaced by another amino acids, or
amino acid Q438 is replaced by another amino acid and the compound comprises amino acids 8255, Y278, R292, E293, L309, Q342, P343, E345, R355, Y373, Q418, V422, H433, H435, T437 and P445, or
amino acids V422 and H435 and optionally one or more amino acids selected from the group consisting of R255, L309, Q342, P343, E345, Y373, H433, and T437 are replaced by another amino acid and the compound comprises amino acids Q418, R355 and P445, or
amino acids Q418, R355 and P445 are replaced by another amino acid and the compound comprises amino acid V422 and H435 and one or more amino acids selected from the group consisting of R255, L309, Q342, P343, E345, Y373, H433 and T437, or
amino acids H433, T437, Q438 and V422 are replaced by another amino acid and the compound comprises amino acids H435, Q418, R355 and P445, or
amino acids H435, Q418, R355 and P445 are replaced by another amino acid and the compound comprises amino acids H433, T437, Q438 and V422.

11. The method according to claim 1, comprising contacting said sample with at least two compounds comprising a recombinant human IgG class Fc domain, the method further comprising determining binding of RF to said at least two compounds, wherein:
a first compound A comprises:
a first RF epitope wherein amino acid H435 and one or more amino acids selected from the group consisting of R255, L309, Q342, P343, E345, Y373, H433, T437, and Q438 of said IgG Fc domain are replaced by another amino acid, and
a second RF epitope comprising amino acid V422 and optionally one or more amino acids selected from the group consisting of R355, Q418, Q438, and P445 of said IgG Fc domain, and
a second compound B comprises:
a first RF epitope wherein amino acid V422 and optionally one or more amino acids selected from the group consisting of R355, Q418, Q438, and P445 of said IgG Fc domain are replaced by another amino acid, and
a second RF epitope comprising amino acid H435 and one or more amino acids selected from the group consisting of R255, L309, Q342, P343, E345, Y373, H433, T437, and Q438 of said IgG Fc domain, wherein said amino acid numbering is according to EU numbering.

12. The method according to any one of claims 1-11 wherein the subject is suffering from or suspected of suffering from a disease or condition characterized by the presence of rheumatoid factor.

13. The method according to claim 11 or 12, wherein a ratio of the level of RF that binds to compound A as compared to the level of RF that binds to compound B is determined and the more said ratio deviates from 1, the lower the risk of progression to arthritis.

14. A combination of a first compound A and a second compound B, each compound comprising a human IgG class fragment crystallizable (Fc) domain, wherein
compound A comprises:
a first RF epitope wherein amino acid H435 and one or more amino acids selected from the group consisting of R255, L309, Q342, P343, E345, Y373, H433, T437, and Q438 of said IgG Fc domain are replaced by another amino acid, and
a second RF epitope comprising amino acid V422 and optionally one or more amino acids selected from the group consisting of R355, Q418, Q438, and P445 of said IgG Fc domain, and
compound B comprises:
a first RF epitope wherein amino acid V422 and optionally one or more amino acids selected from the group consisting of R355, Q418, Q438, and P445 of said IgG Fc domain are replaced by another amino acid, and
a second RF epitope comprising amino acid V422 and optionally one or more amino acids selected from the group consisting of R355, Q418, Q438, and P445 of said IgG Fc domain.

15. A combination according to claim 14, wherein said compound A is a compound as defined in any one of claims 3-9 and said compound B is a compound as defined in any one of claims 3-9, with the proviso that said compounds A and B are different.

16. A compound comprising a recombinant human IgG class fragment crystallizable (Fc) domain, wherein said Fc domain comprises:
1) a first RF epitope wherein amino acids H435 and one or more amino acids selected from the group consisting of R255, L309, Q342, P343, E345, Y373, H433, T437, and Q438 of said IgG Fc domain are replaced by another amino acid, and
2) a second RF epitope that comprises amino acid V422 and one or more amino acids selected from the group consisting of R355, Q418 and P445 of said IgG Fc
domain, wherein said amino acid numbering is according to EU numbering.

17. A compound comprising a recombinant human IgG class fragment crystallizable (Fc) domain, wherein said Fc domain comprises:
1) a first RF epitope wherein amino acid V422 and optionally one or more amino acids selected from the group consisting of R355, Q418, Q438, and P445 of said IgG Fc domain are replaced by another amino acid, and
2) a second RF epitope that comprises amino acid H435 and one or more amino acids selected from the group consisting of R255, L309, Q342, P343, E345, Y373, H433 and T437 of said IgG Fc domain, wherein said amino acid numbering is according to EU numbering.

18. A compound comprising a recombinant human IgG class fragment crystallizable (Fc) domain, wherein in said compound amino acids H435 and V422 and one or more amino acids selected from the group consisting of R255, L309, Q342, P343, E345, Y373, H433 and T437 are replaced by another amino acid and the compound comprises one or more amino acids selected from the group consisting of R255, Q342, P343, E345, Y373, H433, T437, R355, Q418 and P445.

19. A compound comprising a recombinant human IgG class fragment crystallizable (Fc) domain, wherein said compound is a compound as defined in any one of claims 3-9.

20. The method, combination or compound according to any one of claims 1 to 19 wherein said compound comprises a recombinant human IgG1, IgG2 or IgG4 class Fc domain, preferably IgG1.

21. The method according to any one of claim 1 to 13 or 20 wherein said determining comprises determining the percentage of RF that binds to said at least one compound as compared to binding to a compound comprising an Fc domain wherein said first and said second RF epitope are both unaltered.

22. Use of a combination or compound according to any one of claims 14-19 in diagnosis, preferably wherein said diagnosis comprises typing rheumatoid factor with a method according to any one of claim 1 to 13, 20 or 21.

23. A method for determining a rheumatoid factor (RF) reactivity profile characteristic for a disease or condition comprising typing RFs in one or more samples, preferably a plurality of samples, from a patient suffering from the disease or condition with a method for typing RF according to any one of claim 1 to 13, 20 or 21.

24. A method for determining a treatment strategy for a subject, comprising typing rheumatoid factor in a sample of the subject with a method for typing RF according to any one of claim 1 to 13, 20 or 21, and determining a treatment strategy for said subject if said typing and/or the established RF reactivity profile indicates that said subject is suffering from or at risk of suffering from a disease or condition characterized by the presence of RF.
